SEQUENCE LISTING (4 sequences)

SEQ ID NO: 1 - protein, 213 aa, Artificial Sequence
  Anti-CD20 Antibody Wild Type Light Chain Amino Acid Sequence
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 2 - protein, 451 aa, Artificial Sequence
  Anti-CD20 Antibody Wild Type Heavy Chain Amino Acid Sequence
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 235 aa, Artificial Sequence
  Light Chain with signal sequence
MDFQVQIISFLLISASVIMSRGQIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 470 aa, Artificial Sequence
  Heavy Chain with signal sequence
MGWSLILLFLVAVATRVLSQVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2014/0004037 A1
(43) Pub. Date: Jan. 2, 2014

(54) STABILIZATION OF THE ANTI-CD20 ANTIBODY RITUXIMAB

(71) Applicant: Rosemarie Wilton, Elmhurst, IL (US)

(72) Inventor: Rosemarie Wilton, Elmhurst, IL (US)

(73) Assignee: Therapeutic Proteins, Inc., Chicago, IL (US)

(21) Appl. No.: 13/354,123

(22) Filed: Jan. 19, 2012

(51) Int. Cl. A61K 39/395; C12N 15/13; C07K 17/00; C07K 17/08; C07K 19/00; C40B 30/04; A61K 51/10; A61P 35/00; C12N 15/63; C12N 5/10; G01N 33/566; C07K 16/28; C07K 17/10

(52) U.S. Cl. 424/1.49; 530/389.6; 536/23.53; 530/391.1; 530/391.3; 530/391.7; 530/362; 424/173.1; 424/178.1; 424/135.1; 424/153.1; 435/320.1; 435/343; 436/501; 435/7.92; 506/9

(57) ABSTRACT

The invention provides isolated stabilized anti-CD20 antibodies and methods of their manufacture and use in diagnosis and treatment animal diseases including human lymphoma, leukemia, and autoimmunity.

### BACKGROUND OF THE INVENTION

Rituximab (a.k.a, IDEC-C2B8, RITUXAN, MABTHERA) is a chimeric anti-CD20 monoclonal antibody that is used to treat Non-Hodgkin's lymphoma, chronic lymphocytic leukemia, rheumatoid arthritis and Wegener's granulomatosis (a form of microscopic polyangiitis.) CD20 is a B lymphocyte-specific cell-surface molecule involved in the regulation of transmembrane Ca²⁺ conductance and cell-cycle progression during human B cell activation. CD20 is first expressed by human pre-B cells in the bone marrow, predominantly after Ig heavy chain rearrangement, with expression persisting until plasma cell differentiation. CD20 is expressed on the surface of 90% of B-cell non-Hodgkin's lymphomas, but the not found on hematopoietic stem cells, pro-B-cells, normal plasma cells or normal non-hemataopoietic tissues. CD20 is not shed from the cell surface and free CD20 antigen is not found in the circulation. Based on in vitro studies, it is currently thought that the rituximab Fc domain recruits immune effector functions to mediate B-cell lysis.

Antibodies are protein molecules produced by the immune system for identifying and neutralizing foreign pathogens such as viruses and bacteria. Because of their exquisite binding sensitivity and specificity, antibodies are valuable reagents which have a wide range of therapeutic and diagnostic uses. Antigens are the molecules that stimulate the synthesis of antibodies in vivo. When bound to antigens can activate a number of processes which can be used to attack neoplastic or autoimmune B-cells including, e.g., the fixation of complement, the phagocytosis of antigen bearing cells, and direct cell mediated cytotoxicity.

However, as is the case with most protein molecules, antibodies need to be maintained within a narrow range of pH, temperature and solvent conditions to retain chemical and biological stability. Furthermore, because of the differences in amino acid sequence which impart each antibody with its unique specificity, individual antibodies vary widely in stability. Methods and compositions have recently been described (see e.g., U.S. patent application Ser. No. 13/002,013; Publication No. 20110130324) which can enhance the stability of antibodies while maintaining their antigen binding activity. Such stabilization of therapeutic antibodies can result in improved serum half-life, lower dosage requirements, reduced side-effects, improved shelf-life and reduced shipping and storage costs.

Accordingly, there remains a long-felt need for stabilized versions of all therapeutic antibodies including rituximab.

### BRIEF SUMMARY OF THE INVENTION

The invention provides isolated stabilized anti-CD20 antibodies, wherein (a) the isolated stabilized anti-CD20 antibody light chain amino acid sequence comprises SEQ ID NO: 1 or a variant comprising SEQ ID NO: 1 with one or more of the following amino acid sequence changes: S41Q, S42P, W46L, W46I, S55P, V59A, V59D, S69D, S69N, Y70F, and A79P; (b) the isolated stabilized anti-CD20 antibody heavy chain amino acid sequence comprises SEQ ID NO: 2 or a variant comprising SEQ ID NO: 2 with one or more of the following amino acid sequence changes: V37L and M20L, M20L and A92G, Q5V, P7S, E10G, M20L, M20I, T28S, Y32S, A68F, A68V, T69I, L70I, A72V, K74N, K74T, M81L, S84N, A92G, N109D, A113Q, V263L, V277I, F279L, and V312I; and (c) wherein the isolated stabilized anti-CD20 antibody light chain amino acid sequence comprises at least one of the amino acid sequence changes in SEQ ID NO: 1 listed in (a) or the stabilized anti-CD20 antibody heavy chain amino acid sequence comprises at least one of the amino acid sequence changes in SEQ ID NO: 2 listed in (b). Said isolated stabilized anti-CD20 antibodies and their individual immunoglobulin light and heavy chains are stabilized in comparison to the “wild type anti-CD20 antibody” and its corresponding individual immunoglobulin light and heavy chains, wherein the “wild type anti-CD20 antibody” refers to an antibody with the light chain (SEQ ID NO: 1) and heavy chain (SEQ ID NO: 2) sequences (which are based on the Rituximab sequences first reported in U.S. Pat. No. 5,843,439, but with two corrections of the heavy chain sequence included herein).

The invention also provides isolated nucleic acids encoding immunoglobulin heavy or light chains, or both, which comprise said stabilized anti-CD20 antibodies provided by the invention. The invention provides for methods of making said stabilized anti-CD20 antibodies. Accordingly, the invention provides for cells which comprise vectors which express one or both of the immunoglobulin heavy and light chains of said stabilized anti-CD20 antibodies.

The invention provides that isolated stabilized anti-CD20 antibodies in accordance with the invention include, without limitation, antibodies in the form of an intact antibody, a Fv fragment, a single chain variable region (ScFv) antibody, a monoclonal antibody, a Fab antibody fragment, a Fab′ antibody fragment or a Fab′2 Fab antibody fragment.

The invention further provides methods of treating diseases in patients comprising administering to the patient a therapeutically effective amounts of any one or more of said stabilized anti-CD20 antibodies. In preferred embodiments, the invention provides methods of treating Non-Hodgkin's lymphoma, chronic lymphocytic leukemia, rheumatoid arthritis, Wegener's granulomatosis and microscopic polyangiitis with said stabilized anti-CD20 antibodies.

Further, the invention provides methods of quantitatively detecting a CD20 polypeptide in a patient or biological specimen comprising administering to the patient or contacting the specimen with a diagnostically effective amount of a stabilized anti-CD20 antibody provided by the invention.

### BRIEF DESCRIPTION OF THE DRAWINGS

FIG. 1 depicts SEQ ID NO: 1, the wild type anti-CD20 antibody light chain amino acid sequence, without its signal sequence.

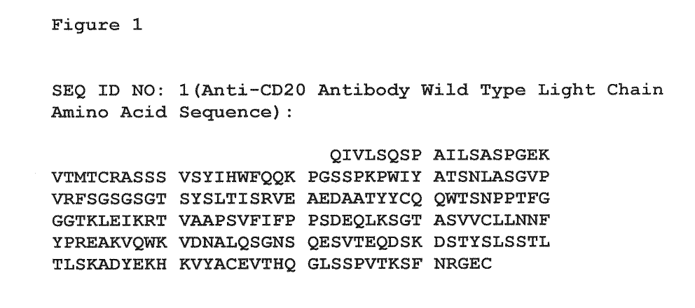

FIG. 2 depicts SEQ ID NO: 2, the wild type anti-CD20 antibody heavy chain amino acid sequence, without its signal sequence. Sequences are based on the anti-CD20 sequence disclosed by U.S. Pat. No. 5,843,439 with two sequence corrections shown by single and double underlines.

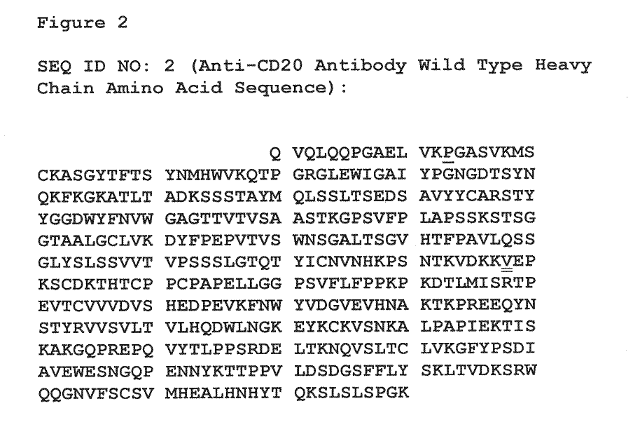

FIG. 3 depicts SEQ ID NO: 3, the wild type anti-CD20 antibody light chain amino acid sequence, with its signal sequence.

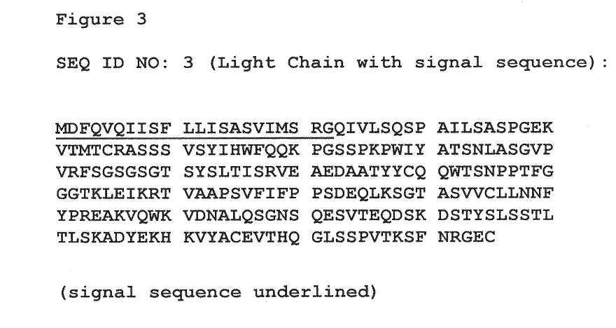

FIG. 4 depicts SEQ ID NO: 4, the wild type anti-CD20 antibody heavy chain amino acid sequence, with its signal sequence.

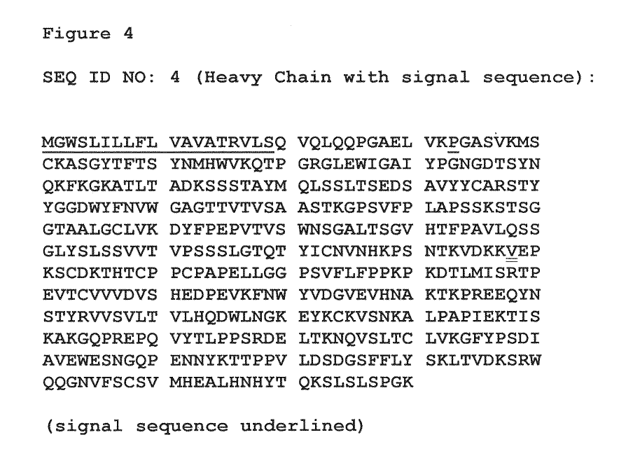

FIG. 5 depicts the results of capillary differential scanning calorimetry analysis of wild type and variant anti-CD20 antibodies. The wild type anti-CD2 is identified as sample RW004. Samples RW005-009 consist of the stabilized variants. The sequence changes and Tₘ values are shown in Table 8.

FIG. 6 depicts the results of Protein A Binding thermal challenge assays.

### DETAILED DESCRIPTION OF THE INVENTION

The present invention relates to certain CD20-binding antibodies which have enhanced stability, the production of said antibodies, and the use of said antibodies.

### DEFINITIONS

“Isolated” and “purified” are used interchangeably herein and refer to a molecule in a state where it is substantially separated from other biologic molecules such as proteins, nucleic acids, lipids, and polysaccharides. This is in contrast to such a molecule's normal in vivo state where it exists in the presence of a huge number of other molecules.

As used herein, the terms “stabilized,” “stabilized protein,” and “stabilized polypeptide” “are used interchangeably herein and refer to a protein in which amino acid changes have been made which render the protein more resistant to conditions such as heat, cold, vibration, tonicity and the like which tend to decrease the protein's normal function in comparison to a wild type protein (i.e., the protein without said amino acid changes.) For example, the stability of a protein is related to its Gibbs free energy of unfolding, ΔGu, which is temperature-dependent. The stability of most proteins decreases with temperature; as the temperature increases, the AGu decreases and becomes zero at equilibrium where the concentrations of folded and unfolded protein are equal. At this point, the temperature is considered as “melting temperature” (Tₘ). Thermal unfolding of proteins in the presence of fluorescent dyes can be used to assess protein Tₘ. Although not an equilibrium method, the technique can be used to rank proteins according to relative Tₘ. (see also, e.g. Niesen F. H., Berglund H. and Vedadi M.: The use of differential scanning fluorimetry to detect ligand interactions that promote protein stability. Nature Protocols 2007, 2:2212-21.) Stabilized proteins in accordance with the current invention will have, e.g., a Tₘ which is at least 0.3° C. higher than the wild type Tₘ, preferably a Tₘ which is at least 0.3° C. higher than the wild type Tₘ, more preferably a Tₘ which is at least 0.5° C. higher than the wild type Tₘ, even more a Tm which is at least 1.0° C. higher than the wild type Tm, and even more preferably a Tₘ which is at least 5.0° C. higher than the wild type Tₘ, where Tₘ is determined by any suitable method known to those of ordinary skill in the art.

“Polypeptide,” “peptide,” and “protein” are used interchangeably herein and refer to a compound made up of a chain of amino acid residues linked by peptide bonds. An “active portion” of a polypeptide means a peptide that is less than the full length polypeptide, but which retains measurable biological activity and retains biological detection.

As used herein, the term “tumor” refers to any neoplastic growth, proliferation or cell mass whether benign or malignant (cancerous), whether a primary site lesion or metastases.

As used herein, the term “cancer” refers to a proliferative disorder caused or characterized by a proliferation of cells which have lost susceptibility to normal growth control. Cancers of the same tissue type usually originate in the same tissue, and may be divided into different subtypes based on their biological characteristics. Four general categories of cancer are carcinoma (epithelial cell derived), sarcoma (connective tissue or mesodermal derived), leukemia (blood-forming tissue derived) and lymphoma (lymph tissue derived). Cancer may involve every organ and tissue of the body may be affected. Specific examples of cancers that do not limit the definition of cancer may include melanoma, leukemia, astrocytoma, glioblastoma, retinoblastoma, lymphoma, glioma, Hodgkin's lymphoma, and chronic lymphocytic leukemia. Examples of organs and tissues that may be affected by various cancers include pancreas, breast, thyroid, ovary, uterus, testis, prostate, pituitary gland, adrenal gland, kidney, stomach, esophagus, rectum, small intestine, colon, liver, gall bladder, head and neck, tongue, mouth, eye and orbit, bone, joints, brain, nervous system, skin, blood, nasopharyngeal tissue, lung, larynx, urinary tract, cervix, vagina, exocrine glands, and endocrine glands. Alternatively, a cancer can be multicentric or of unknown primary site (CUPS).

As used herein “tumor targeting antibody” refers to a disease targeting antibody wherein the disease is a lymphoma, leukemia, tumor, cancer, neoplasm or the like.

As used herein “therapeutically effective amount” refers to an amount of a composition that relieves (to some extent, as judged by a ordinarily skilled clinician) one or more symptoms of the disease or condition in a mammal. Additionally, by “therapeutically effective amount” of a composition is meant an amount that returns to normal, either partially or completely, physiological or biochemical parameters associated with or causative of a disease or condition. A clinician skilled in the art can determine the therapeutically effective amount of a composition in order to treat or prevent a particular disease condition, or disorder when it is administered, such as intravenously, subcutaneously, intraperitoneally, orally, or through inhalation. The precise amount of the composition required to be therapeutically effective will depend upon numerous factors, e.g. such as the specific activity of the active agent, the delivery device employed, physical characteristics of the agent, purpose for the administration, in addition to many patient specific considerations. But, a determination of a therapeutically effective amount is within the skill of an ordinarily skilled clinician upon the appreciation of the disclosure set forth herein.

The terms “treating,” “treatment,” “therapy.” and “therapeutic treatment” as used herein all refer to curative therapy, prophylactic therapy, or preventative therapy. An example of “preventative therapy” is the lessening the chance of a targeted disease (e.g., cancer or other proliferative disease), or related condition thereto, by at least 5%, preferably by at least 10%, more preferably by at least 15%, and even more preferably by at least 20% Those in need of treatment include those already with the disease or condition as well as those prone to have the disease or condition to be prevented. The terms “treating,” “treatment,” “therapy,” and “therapeutic treatment” as used herein also describe the management and care of a mammal for the purpose of combating a disease, or related condition, and includes the administration of a composition to alleviate the symptoms, side effects, or other complications of the disease, condition. Therapeutic treatment for cancer includes, but is not limited to, surgery, chemotherapy, radiation therapy, gene therapy, and immunotherapy.

As used herein, the terms “effector,” “agent” or “drug” or “therapeutic agent” refers to a chemical agent, a mixture of chemical compounds, a biological macromolecule, or an extract made from biological materials such as bacteria, plants, fungi, or animal (particularly mammalian) cells or tissues that are suspected of having therapeutic or medicinal properties. The agent or drug can be purified, substantially purified or partially purified. An “agent” according to the present invention, also includes a radiation therapy agent or a “chemotherapeutic agent” (e.g., a small molecule drug.)

As used herein, the term “diagnostic agent” refers to agents allowing for the detection and/or quantitation of CD20, e.g., plasma/circulating CD20 by any suitable method such as, e.g., immunohistology or flow cytrometry.

As used herein, the term “chemotherapeutic agent” refers to a chemical agent with activity against cancer, neoplastic, and/or proliferative diseases (e.g., a small molecule drug.)

As used herein, the term “radiotherapeutic regimen” or “radiotherapy” refers to the administration of radiation to kill cancerous cells. Radiation interacts with various molecules within the cell, but the primary target, which results in cell death is the deoxyribonucleic acid (DNA). However, radiotherapy often also results in damage to the cellular and nuclear membranes and other organelles. DNA damage usually involves single and double strand breaks in the sugar-phosphate backbone. Furthermore, there can be cross-linking of DNA and proteins, which can disrupt cell function. Depending on the radiation type, the mechanism of DNA damage may vary as does the relative biologic effectiveness. For example, heavy particles (i.e., protons, neutrons) damage DNA directly and have a greater relative biologic effectiveness. Whereas, electromagnetic radiation results in indirect ionization acting through short-lived, hydroxyl free radicals produced primarily by the ionization of cellular water. Clinical applications of radiation consist of external beam radiation (from an outside source) and brachytherapy (using a source of radiation implanted or inserted into the patient). External beam radiation consists of X-rays and/or gamma rays, while brachytherapy employs radioactive nuclei that decay and emit alpha particles, or beta particles along with a gamma ray.

As used herein the terms “biologics,” “biologicals,” “biological agents,” “alternative therapeutic regimen” or “alternative therapy” are therapies other than surgery, chemotherapy (small molecule drugs) or radiation therapy, including e.g., receptor tyrosine kinase inhibitors (for example Iressa™ (gefitinib), Tarceva™ (erlotinib), Erbitux™ (cetuximab), imatinib mesilate (Gleevec™)); proteosome inhibitors (for example bortezomib (Velcade™)); VEGFR2 inhibitors such as PTK787 (ZK222584), aurora kinase inhibitors (for example ZM447439); mammalian target of rapamycin (mTOR) inhibitors, cyclooxygenase-2 (COX-2) inhibitors, rapamycin inhibitors (for example sirolimus (Rapamune™)); farnesyltransferase inhibitors (e.g., tipifamib (Zarnestra™)); matrix metalloproteinase inhibitors (for example BAY 12-9566; sulfated polysaccharide tecogalan); angiogenesis inhibitors (for example Avastin™ (bevacizumab); analogues of fumagillin such as TNP-4; carboxyaminotriazole; BB-94 and BB-2516; thalidomide; interleukin-12; linomide; peptide fragments; and antibodies to vascular growth factors and vascular growth factor receptors; platelet derived growth factor receptor inhibitors, protein kinase C inhibitors, mitogen-activated kinase inhibitors, mitogen-activated protein kinase inhibitors, Rouse sarcoma virus transforming oncogene (SRC) inhibitors, histonedeacetylase inhibitors, small hypoxia-inducible factor inhibitors, hedgehog inhibitors, TGF-β signaling inhibitors, and the like.

In keeping with the preceding, an immunotherapeutic agent would also be considered an alternative therapeutic regimen. For example, serum or gamma globulin containing preformed antibodies; nonspecific immunostimulating adjuvants; active specific immunotherapy; and adoptive immunotherapy. In addition, alternative therapies may include other biological-based chemical entities such as polynucleotides, including antisense molecules, polypeptides, antibodies, gene therapy vectors and the like. Such alternative therapeutics may be administered alone or in combination with other therapeutic regimens described herein. Methods of use of chemotherapeutic agents and other agents used in alternative therapeutic regimens in combination therapies, including dosing and administration regimens, will also be known to a one skilled in the art.

As used herein the terms “tumor localization” or “disease localization,” as used herein refer to the degree to which, upon injection into a tumor bearing animal, an anti-CD20 antibody collects or concentrates in or at the site of a CD20-expressing tumor. Tumor localization may be measured by any suitable method including, but not limited to, labeling the antibody with a fluorescent dye, injecting the now fluorescent antibody into an animal with a tumor and determining a ratio of tumor fluorescence to the fluorescence from skin or other tissue away from any gross tumor, wherein localization is present if said ratio is >20, preferably >10, more preferably >5.

As used herein, the term “vector” means any polynucleotide which can be comprised of a sequence encoding an anti-CD20 antibody light or heavy chain or portion thereof (i.e., the “coding sequences” are cloned into the vector) and can be used for the transformation of cells (e.g., by DNA transfection, such that that the polynucleotide can replicate in that cell and, e.g. the presence of said polynucleotide can be selected for by an antibiotic.) Accordingly, a vector can be said to be “capable of expressing” a gene cloned into that vector if the vector includes control sequences (e.g., promoters, enhancers, while lacking repressors) which can direct the transcription of the cloned gene. As used herein, when it is said that “expression is inducible” it is meant that the transcription of the vector sequences encoding an anti-CD20 Antibody or portion thereof can be controlled such that said transcription can be turned on or off. Cells may be said to be “transiently transformed” with a vector if the vector sequences are maintained in the cell for a limited period of time (e.g., days) or “stably transformed” if the vector sequences are maintained in the cell indefinitely (particularly if it is selected for.)

As used herein the term “sequence changes” means an alteration in the amino acid or nucleic acid sequence (i.e., a “mutation”).

Anti-CD20 Antibodies

Generally, antibodies are composed of two light chains and two heavy chain molecules; these chains form a general “Y” shape, with both light and heavy chains forming the arms of the Y and the heavy chains forming the base of the Y. Light and heavy chains are divided into domains of structural and functional homology. The variable domains of both the light (“V_{L}”) and the heavy (“V_{H}”) chains determine recognition and specificity. The constant region domains of light (“C_{L}”) and heavy (“C_{H}”) chains confer important biological properties, e.g., antibody chain association, secretion, transplacental mobility, Fc receptor binding, complement fixation, opsinization, activating antibody dependent cellular cytotoxicity (“ADCC”), and the like. The series of events leading to immunoglobulin gene expression in the antibody producing cells are complex. The variable domain region gene sequences are located in separate germ line gene segments referred to as “V_{H},” “D_{H},” and “J_{H},” or “V_{H}” and “J_{L}.” These gene segments are joined by DNA rearrangements to form the complete V regions expressed in heavy and light chains, respectively. The rearranged, joined V segments (V_{L}-J_{L} and V_{H}-D_{H}-J_{H}) then encode the complete variable regions or antigen binding domains of light and heavy chains, respectively. (As used herein the terms “Immunoglobulin heavy and light chains,” “antibody heavy and light chains,” and “heavy and light chains” are interchangeable.)

As used herein, the term “anti-CD20 antibody” is an antibody which specifically recognizes a cell surface non-glycosylated phosphoprotein of 35,000 Daltons, typically designated as the human B lymphocyte restricted differentiation antigen Bp35, commonly referred to as “CD20.” As used herein the term “wild type anti-CD20 antibody” means a CD20 binding antibody with the immunoglobulin light chain amino acid sequence of SEQ ID NO: 1 and the immunoglobulin heavy chain amino acid sequence of SEQ ID NO: 2 or their equivalents in the form of Fv fragments, single chain variable region (ScFv) antibodies, Fab antibody fragments, Fab′ antibody fragments or Fab′2 Fab antibody fragments.

As used herein, the term “chimeric” refers to antibodies which comprise portions from two or more different species (e.g., mouse and human). In particular, when used in reference to anti-CD20 antibodies, the term encompasses antibodies which are most preferably derived using recombinant deoxyribonucleic acid techniques and which comprise both human (including immunologically “related” species, e.g., chimpanzee) and non-human components: the constant region of the chimeric antibody is substantially identical to the constant region of a natural human antibody; the variable region of the chimeric antibody is derived from a non-human source and has the desired antigenic specificity to the CD20 cell surface antigen.

As used herein, the phrase “immunologically active” when used in reference to chimeric anti-CD20 antibodies, means an antibody which binds human C1q, fixes complement, opsinizes surface CD20 bearing cells, mediates complement dependent lysis (“CDC”) of human B lymphoid cell lines, and lyses human target cells through antibody dependent cellular cytotoxicity (“ADCC”).

Rituximab is a complete antibody molecule which has an approximate molecular weight of 145 kD and a CD20 binding affinity of approximately 8.0 nM. Said antibody binding affinity can be determined by any suitable method known to those of ordinary skill. For example, the strength of antibody binding to its antigen can be assessed by radioimmune assay (RIA), ELISA, or binding in a column support format. Dissociation is performed by increasing denaturating conditions, or by competition with a related or cold antigen. Thle dissociation constant, Kd, can be determined by a Scatchard plot (see, e.g., U.S. Pat. Nos. 5,843,439 and 8,057,793.)

The recently described methods for systematic improvement of protein stability have been disclosed in U.S. patent application Ser. No. 13/002,013 (Patent Application Publication No. 20110130324) have applied to antibody molecules with the stabilization of single chain Fvs (scFvs). In particular, using this approach amino acid sequence changes can be made in antibody light and heavy chains that result in stabilization without significantly effecting antigen binding. This is possible because the method focuses on the framework regions of the antibody in order to avoid modifications of its binding properties. The current invention demonstrates, for the first time, application of this stabilization approach to a full-length therapeutic monoclonal antibody. This polypeptide stabilization technology utilizes a proprietary method to identify amino acids changes that are likely to improve the stability of an antibody without affecting its binding function. In general terms this protein stabilization method is based on (1) evaluation of the target sequence using a proprietary database of antibody domain variants for which thermal and thermodynamic stability have been characterized and (2) identification of sequence variations occurring in functional, homologous proteins.

Accordingly, the invention provides isolated stabilized anti-CD20 antibodies and methods of their manufacture and use, wherein (a) the isolated stabilized anti-CD20 antibody light chain amino acid sequence, or the equivalent of the light chain amino acid sequence, comprises SEQ ID NO: 1 or SEQ ID NO: 1 with one or more of the following amino acid sequence changes: S41Q, S42P, W46L, W46L, S55P, V59A, V59D, S69D, S69N, Y70F, and A79P; (b) the isolated stabilized anti-CD20 antibody heavy chain amino acid sequence, or the equivalent of the heavy chain amino acid sequence, comprises SEQ ID NO: 2 or SEQ ID NO: 2 with one or more of the following amino acid sequence changes: V37L and M20L, M20L and A92G, Q5V, P7S, E10G, M20L, M20I, T28S, Y32S, A68F, A68V, T69I, L70I, A72V, K74N, K74T, M81L, S84N, A92G, N109D, A113Q, V263L, V277I, F279L and V312I; (c) the isolated stabilized anti-CD20 antibody light chain amino acid sequence, or the equivalent of the light chain amino acid sequence, comprises with at least one of the amino acid sequence changes in SEQ ID NO: 1 listed in (a) or the stabilized anti-CD20 antibody heavy chain amino acid sequence, or the equivalent of the heavy chain amino acid sequence, comprises at least one of the amino acid sequence changes in SEQ ID NO: 2 listed in (b); and (d) the isolated stabilized anti-CD20 antibody is in the form of an intact antibody, a Fv fragment, a single chain variable region (ScFv) antibody, a monoclonal antibody, a Fab antibody fragment, a Fab′ antibody fragment or a Fab′2 Fab antibody fragment. By “Fv fragment” it is meant any combination of shortened heavy and light chains held together by any suitable means, including e.g., disulfide bonds, an amino acid linker sequence or a nonpeptide molecule coupled to both chains.

By the “the equivalent” of the light or heavy chain amino acid sequences set forth by SEQ ID NOs: 1 and 2, respectively, it is meant the amino acid residue in the sequence of a Fv fragment, single chain variable region (ScFv) antibody, Fab antibody fragment, Fab′ antibody fragment or Fab′2 Fab antibody fragment in question, which based on local sequence alignment and in view of its Kabat or EU residue number (See Tables 1-3), corresponds to the an amino acid change based on SEQ ID NO: 1 or 2 as disclosed herein.

The invention provides isolated stabilized anti-CD20 antibodies and methods of their manufacture and use, wherein the light chain amino acid sequence comprises SEQ ID NO: 1 with a W46L amino acid change, as well as isolated stabilized anti-CD20 antibodies wherein the heavy chain amino acid sequence comprises SEQ ID NO: 2 with one of the following sequence changes M20L, M20I, M81L, A92G, N109D or V263L.

The invention also provides isolated stabilized anti-CD20 antibodies and methods of their manufacture and use, wherein the light chain amino acid sequence comprises SEQ ID NO: 1 with or without W46L or W46I amino acid changes and the heavy chain amino acid sequence comprises SEQ ID NO: 2 with M20L, M20I, M81L, A92G, N109D or V263L amino acid changes; the heavy chain amino acid sequence comprises SEQ ID NO: 2 with M20L, M20I, M81L, A92G, and N109D amino acid changes; and the heavy chain amino acid sequence comprises SEQ ID NO: 2 with M20L, M20I, M81L, and A92G amino acid changes.

The invention further provides isolated stabilized anti-CD20 antibodies and methods of their manufacture and use, wherein, the antibody is considered stabilized when either its heavy or light chain has superior thermal stability to the corresponding heavy or light chain of wild type anti-CD20 antibody as measured by any suitable method known to those of ordinary skill, including e.g., differential scanning calorimetry (DSC), circular dichroism (CD) spectroscopy, fluorescence emission spectroscopy, nuclear magnetic resonance (NMR) spectroscopy, size exclusion chromatography or a thermal challenge assay.

In other embodiments, the invention provides stabilized anti-CD20 antibodies and methods of their manufacture and use, wherein as a result of amino acid sequence changes resulting in the anti-CD20 antibody having enhanced antigen binding activity, shelf life, serum half life, AUC or Cₘₐₓ when compared to a the same quantity of wild type anti-CD20 antibody.

The invention provides isolated nucleic acids encoding the light or heavy chains of the stabilized anti-CD20 antibodies described herein, including e.g., wherein the nucleic acid is an RNA or DNA. The invention also provides isolated vectors that direct the expression of any one or more of said nucleic acids (i.e., mono, bi or polycistronic vectors), including e.g., wherein said expression is inducible. Accordingly, invention provides prokaryotic and eukaryotic cells comprising one or more of these vectors, including wherein the cell is a Chinese hamster ovary cell. Said cells can be stably or transiently transformed with said vectors and the expression of the anti-CD20 light and heavy chains can be constitutive or inducible.

The invention provides isolated anti-CD20 antibodies and methods of their manufacture and use, wherein the anti-CD20 antibody light or heavy chain amino acid sequences have further amino acid sequence changes that enhance the antibody's ability to bind to the CD20 molecule, fix complement, opsinize a CD-20 expressing cell, activate antibody dependent cellular cytotoxicity (ADCC), activate antibody dependent programmed cell death, activate macrophage dependent anti-CD20 immune responses. CD20 bearing cells sensitivity to chemotherapy or reduce the anti-CD20 antibody's ability to fix complement and combinations thereof (see, e.g., U.S. Pat. No. 8,084,582.)

The invention provides isolated stabilized anti-CD20 antibodies and methods of their manufacture and use, wherein the anti-CD20 antibody is coupled to an effector, including e.g., wherein the effector is a radioisotope, chemotherapeutic agent, toxin, biologic response modifier or second antibody. The isolated anti-CD20 antibodies of the invention also include anti-CD20 antibodies coupled to PEG, albumin, or polysialic acid and methods of their manufacture and use. Accordingly, the invention provides for pharmacologic compositions comprising any one of the stabilized anti-CD20 antibodies described herein and a pharmaceutically acceptable carrier.

In order to avoid immunogenicity and immune response, it is often preferable to use a humanized CD20 binding antibody or suitable fragments such as Fab′, Fab, or Fab2. Humanized antibody or fragments thereof can be produced by any suitable method, including e.g.: 1) a humanized antibody can be constructed using human IgG backbone replacing the variable CDR region with that of an antibody against CD20, where the heavy and light chain are independently expressed under separate promoters or coexpressed under one promoter with an IRES sequence; 2) a humanized monoclonal antibody can be raised against CD20 using a mouse engineered to have a human immune system; 3) a humanized antibody against CD20 can be raised using phagemid (M13, lambda coliphage, or any phage system capable of surface presentation). The expression of complete antibodies can be accomplished by the coexpression of the heavy chain and light chain in mammalian cells such as, e.g., CHO or 293 cells. Similarly, Fab′, Fab, or Fab2 fragments and single chain antibodies can be prepared using any suitable method known to those of ordinary skill.

There are four general steps to humanize a monoclonal antibody. These are: (1) determining the nucleotide and predicted amino acid sequence of the starting antibody light and heavy variable domains (2) designing the humanized antibody, i.e., deciding which antibody framework region to use during the humanizing process (3) the actual humanizing methodologies/techniques and (4) the transfection and expression of the humanized antibody. See, for example, U.S. Pat. Nos. 4,816,567; 5,807,715; 5,866,692; 6,331,415; 5,530,101; 5,693,761; 5,693,762; 5,585,089; 6,180,370; and 6,548,640 (which are hereby incorporated by reference.) For example, the constant region may be engineered to more resemble human constant regions to avoid immune response if the antibody is used in clinical trials and treatments in humans. See, for example, U.S. Pat. Nos. 5,997,867 and 5,866,692 (which are hereby incorporated by reference.)

Alternatively, antibodies may be screened and made recombinantly by phage display technology. See, for example, U.S. Pat. Nos. 5,565,332; 5,580,717; 5,733,743 and 6,265,150 (which are hereby incorporated by reference.) Alternatively, the phage display technology (McCafferty et al., Nature 348:552-553 (1990)) can be used to produce human antibodies and antibody fragments in vitro.

The Generation of Stabilized Anti-CD20 Antibodies

The stabilized anti-CD20 antibodies provided by the invention were generated by “site-directed mutagenesis.” Site-directed mutagenesis is an established recombinant DNA technology which allows for the change of a DNA sequence at a specific site to generate a pre-determined new sequence. Any suitable site-directed mutagenesis technique may be used in accordance with the current invention. One exemplary suitable technique for site-directed mutagenesis is oligonucleotide mismatch mutagenesis. This technique uses in vitro DNA synthesis to introduce a predetermined single nucleotide change into a cloned gene. The general approach involves cloning the gene or cDNA into an M13 or phagemid vector which permits recovery of single-stranded recombinant DNA. A mutagenic oligonucleotide is then designed whose sequence is complementary to the gene sequence in the region to be mutated, but which has a single nucleotide difference, the intended mutation site. The mutagenic oligonucleotide acts to prime new DNA synthesis resulting in a complementary full-length sequence containing the desired mutation. Mutant and wild type sequences are allowed to anneal forming heteroduplexes. These heteroduplex is used to transform cells, and the desired mutant genes can be identified by screening for the mutation.

Site-directed mutagenesis by PCR is well known to those of ordinary skill in the art and there are numerous suitable strategies which enable base substitutions; deletions and insertions. One such suitable PCR technique for site-directed mutagenesis employs modified PCR primers contain the desired sequence change. The PCR primer sequence simply replaces the original sequence (as long as the changes are minimal enough to allow the primer to anneal to the intended target) (see, e.g., Kadowaki H et al.: “Use of polymerase chain reaction catalysed by Taq DNA Polymerase for site-specific mutagenesis,” Gene (1989) 76(1):161-166).

“Overlap extension PCR” is another suitable and exemplary technique for site-directed mutagenesis uses nested PCR primers to mutate a target region. Complementary PCR primers and the polymerase chain reaction are used to generate two DNA fragments having overlapping ends. These fragments are combined in a subsequent ‘fusion’ reaction in which the overlapping ends anneal, allowing the 3′ overlap of each strand to serve as a primer for the 3′ extension of the complementary strand. The resulting fusion product is amplified further by PCR. Specific alterations in the nucleotide sequence can be introduced by incorporating nucleotide changes into the overlapping primers. (see, e.g., Ho S N et al.: “Site-directed mutagenesis by overlap extension using the polymerase chain reaction,” Gene (1989) 77(1):51-59.)

Yet another suitable and exemplary technique for site-directed mutagenesis uses “inverse PCR.” This technique is used for mutating plasmids. It employs two back-to-back primers to amlplify the whole plusmid and the linear product is then ligated back to the circular form. The primer binding regions can be changed by altering the primer sequences to contain the desired mutation. (see, e.g., Hemsley A et al.: “A simple method for site-directed mutagenesis using the polymerase chain reaction.” Nucleic Acids Res. (1989) 17(16):6545-6551.)

Any suitable polynucleotides with a nucleic acid sequence encoding polypeptides comprising the amino acid sequence of SEQ ID NOs: 1 or 2 or portions of SEQ ID NOs: 1 or 2 may be used as the starting material for the site-directed mutagenesis in accordance with the invention. The polynucleotides with the nucleic acid sequences encoding polypeptides comprising the amino acid sequence of SEQ ID NOs: 1 or 2 or portions of SEQ ID NOs: 1 or 2 disclosed in U.S. Pat. No. 5,843,439 (which is hereby incorporated by reference as if it were set forth in its entirety herein) are well suited as the starting material for the site-directed mutagenesis in accordance with the invention.

Evaluating Thermal Stability

Thermal stability can be evaluated by measuring the melting temperature (Tm) of a composition of the invention using any suitable technique. The melting temperature is the temperature at the midpoint of a thermal transition curve wherein 50% of molecules of a composition are in a folded state.

Thermal stability can be evaluated by calorimetry. An exemplary calorimetric method is Differential Scanning Calorimetry (DSC). DSC employs a calorimeter which is sensitive to the heat absorbances that accompany the unfolding of most proteins or protein domains (see, e.g., Sanchez-Ruiz, et al., Biochemistry, 27: 1648-52, 1988, which is hereby incorporated by reference). To determine the thermal stability of a protein, a sample of the protein is inserted into the calorimeter and the temperature is raised until the antibody, light or heavy chain unfolds. The temperature at which the protein unfolds is indicative of overall protein stability.

Thermal stability can be evaluated by analytical spectroscopy. An exemplary analytical spectroscopy method is Circular Dichroism (CD) spectroscopy. CD spectrometry measures the optical activity of a composition as a function of increasing temperature. Circular dichroism (CD) spectroscopy measures differences in the absorption of left-handed polarized light versus right-handed polarized light which arise due to structural asymmetry. A disordered or unfolded structure results in a CD spectrum very different from that of an ordered or folded structure. The CD spectrum reflects the sensitivity of the proteins to the denaturing effects of increasing temperature and is therefore indicative of a protein's thermal stability (see, e.g., van Mierlo and Steemsma, J. Biotechnol., 79(3):281-98, 2000).

Another exemplary analytical spectroscopy method for measuring thermal stability is Fluorescence Emission Spectroscopy. Fluorescence-based methods to evaluate thermal stability monitor changes in the fluorescence of intrinsic fluorophores (e.g. tryptophan and tyrosine amino acids) or extrinsic fluorophores (e.g. ANS or SYPRO Orange) upon thermal unfolding. (see, e.g., Niesen F. H., Berglund H. and Vedadi M.: The use of differential scanning fluorimetry to detect ligand interactions that promote protein stability. Nature Protocols 2007, 2:2212-21.) Yet another exemplary analytical spectroscopy method for measuring antibody, light or heavy chain thermal stability is Nuclear Magnetic Resonance (NMR) spectroscopy (see, e.g., van Mierlo and Steemsma, J. Biotechnol., 79(3):281-98, 2000).

The thermal stability of a composition of the can also be measured biochemically. An exemplary biochemical method for assessing thermal stability is a thermal challenge assay. In a “thermal challenge assay”, a composition of the invention is subjected to a range of elevated temperatures for a set period of time. For example, test antibody, light or heavy chain molecules, scFv molecules or molecules comprising scFv molecules can be subjected to an range of increasing temperatures, e.g., for 0.1-1.5 hours. The activity of the protein is then assayed by a relevant biochemical assay. For example, if the protein is a binding protein (e.g., binding to Protein A) the binding activity of the binding protein may be determined by a functional assay or quantitative ELISA.

In addition, such an assay may be done in a high-throughput format. For example, such an embodiment, a library of scFv variants may be created using methods known in the art. scFv expression may be induced and scFvs may be subjected to thermal challenge. The challenged test samples may be assayed for binding and those scFvs which are stable may be scaled up and further characterized.

In related technologies, thermal stability is evaluated by measuring the specific heat or heat capacity (Cp) of a composition of the invention using an analytical calorimetric technique (e.g., DSC). The specific heat of a composition is the energy (e.g., in kcal/mol) required to raise by 1° C., the temperature of 1 mol of water. As large Cp is a hallmark of a denatured or inactive protein composition. The change in heat capacity (δCp) of a composition is measured by determining the specific heat of a composition before and after its thermal transition. In other embodiments, thermal stability may be evaluated by measuring or determining other parameters of thermodynamic stability including Gibbs free energy of unfolding (δG), enthalpy of unfolding (δH), or entropy of unfolding (δS):

Manufacture of Stabilized Anti-CD20 Antibodies

The stabilized anti-CD20 antibodies provided by the present invention can be synthesized, detected, quantified and purified using technologies which are well known to those of ordinary skill in the art. For example, cells expressing the polypeptides making up the light and heavy chains of stabilized anti-CD20 antibodies can be generated by placing a cDNA under the control of strong promoter/translation start signal in a vector transfected or transformed into suitable prokaryotic or eukaryotic cells to drive the expression of the polypeptides making up the light and heavy chains of stabilized anti-CD20 antibodies by methods well known to those of ordinary skill in the art.

Alternatively, the polypeptides making up the light and heavy chains of stabilized anti-CD20 antibodies can be made chemically by methods well known to those of ordinary skill in the art. The polypeptides making up the light and heavy chains of stabilized anti-CD20 antibodies can be prepared by standard solid phase synthesis. As is generally known to those of ordinary skill in the art, peptides of the requisite length can be prepared using commercially available equipment and reagents following the manufacturers' instructions for blocking interfering groups, protecting the amino acid to be reacted, coupling, deprotection, and capping of unreacted residues. For example, light and heavy chain peptides can be synthesized using standard automated solid-phase synthesis protocols employing t-butoxycarbonyl-alpha-amino acids with appropriate side-chain protection. Completed peptide is removed from the solid phase support with simultaneous side-chain deprotection using the standard hydrogen fluoride method. Crude peptides are further purified by semi-preparative reverse phase-HPLC (Vydac C18) using acetonitrile gradients in 0.1% trifluoroacetic acid (TFA). The peptides are vacuum dried to remove acetonitrile and lyophilized from a solution of 0.1% TFA in water. Purity can be verified by analytical RP-HPLC. The peptides can be lyophilized and then solubilized in either water or 0.01 M acetic acid at concentrations of 1-2 mg/mL by weight. Suitable equipment can be obtained, for example, from Applied BioSystems, Foster City, Calif., or Biosearch Corporation in San Raphael, Calif.

The invention also provides for a recombinant vector comprising the elements controlling the expression of a polynucleotide sequence encoding one or both of the polypeptides making up the light and heavy chains of stabilized anti-CD20 antibodies. In addition, the invention provides for a cell comprising a nucleic acid encoding such a polypeptide, wherein the cell is a prokaryotic cell or a eukaryotic cell. Methods of microbial and tissue culture are well known to the skilled artisan (see, e.g., Sambrook & Russell, Molecular Cloning: A Laboratory Manual, Cold Spring Harbor Laboratory Press, New York (2001), pp. 16.1-16.54). The invention thus provides for method of making the polypeptides making up the light and heavy chains of stabilized anti-CD20 antibodies comprising: (a) transforming cells with a nucleic acid encoding the light and/or heavy chain polypeptide of a stabilized anti-CD20 antibody; (b) inducing the expression of these polypeptide by the transformed cells; (c) purifying the polypeptide; and (d) assembling a functional antibody.

Polypeptide expression is dependent on the level of RNA transcription, which is in turn regulated by DNA signals. Similarly, translation of mRNA requires, at the very least, an AUG initiation codon, which is usually located within 10 to 100 nucleotides of the 5′ end of the coding sequence. Sequences flanking the AUG initiator codon have been shown to influence its recognition. For example, for recognition by eukaryotic ribosomes, AUG initiator codons embedded in sequences in conformity to a perfect “Kozak consensus” sequence result in optimal translation (see, e.g., Kozak, J. Molec. Biol. 196: 947-950 (1987)). Also, successful expression of an exogenous nucleic acid in a cell can require post-translational modification of a resultant protein.

The nucleic acid molecules described herein preferably comprise a coding region operatively linked to a suitable promoter, for example, a promoter functional in eukaryotic cells. Viral promoters, such as, without limitation, the RSV promoter and the adenovirus major late promoter can be used in the invention. Suitable non-viral promoters include, but are not limited to, the phosphoglycerokinase (PGK) promoter and the elongation factor 1α promoter. Non-viral promoters are desirably derived from the species of the host cells. Additional suitable genetic elements, many of which are known in the artl, also can be attached to, or inserted into the inventive nucleic acid and constructs to provide additional functions, level of expression, or pattern of expression.

In addition, the nucleic acid molecules described herein may be operatively linked to enhancers to facilitate transcription. Enhancers are cis-acting elements of DNA that stimulate the transcription of adjacent genes. Examples of enhancers which confer a high level of transcription on linked genes in a number of different cell types from many species include, without limitation, the enhancers from SV40 and the RSV-LTR. Such enhancers can be combined with other enhancers which have cell type-specific effects, or any enhancer may be used alone.

To optimize protein production in eukaryotic cells, the inventive nucleic acid molecule can further comprise a polyadenylation site following the coding region of the nucleic acid molecule. If desired, the exogenous-nucleic acid also can incorporate splice sites (i.e., splice acceptor and splice donor sites) to facilitate mRNA production while maintaining an inframe, full length transcript. Moreover, the inventive nucleic acid molecules can further comprise the appropriate sequences for processing, secretion, intracellular localization, and the like.

The nucleic acid molecules can be inserted into any suitable vector. Suitable vectors include, without limitation, viral vectors. Suitable viral vectors include, without limitation, retroviral vectors, alphaviral, vaccinial, adenoviral, adeno associated viral, herpes viral, and fowl pox viral vectors. The vectors preferably have a native or engineered capacity to transform eukaryotic cells, e.g., CHO-K1 cells. Additionally, the vectors useful in the context of the invention can be “naked” nucleic acid vectors such as plasmids or episomes, or the vectors can be complexed with other molecules. Other molecules that can be suitably combined with the inventive nucleic acids include without limitation viral coats, cationic lipids, liposomes, polyamines, gold particles, and targeting moieties such as ligands, receptors, or antibodies that target: cellular molecules.

The nucleic acid molecules described herein can be transformed into any suitable cell, typically a eukaryotic cell, such as, e.g., CHO, HEK293, or BHK, desirably resulting in the expression of a light or heavy chain polypeptide such as, e.g., polypeptide comprising of SEQ ID NO: 1 or 2 or a variant or homolog thereof as described herein. The cell can be cultured to provide for the expression of the nucleic acid molecule.

Accordingly, the invention provides for a cell transformed or transfected with an inventive nucleic acid molecule described herein. Means of transforming, or transfecting, cells with exogenous DNA molecules are well known in the art. For example, without limitation, a DNA molecule is introduced into a cell using standard transformation or transfection techniques well known in the art such as calcium-phosphate or DEAE-dextran-mediated transfection, protoblast fusion, electroporation, liposomes, cationic lipid, and direct microinjection (see, e.g., Sambrook & Russell, Molecular Cloning: A Laboratory Manual, Cold Spring Harbor Laboratory Press, New York (2001), pp. 1.1-1.162, 15.1-15.53, 16.1-16.54).

Another example of a transformation method is the protoplast fusion method, protoplasts derived from bacteria carrying high numbers of copies of a plasmid of interest are mixed directly with cultured mammalian cells. After fusion of the cell membranes (usually with polyethylene glycol), the contents of the bacteria are delivered into the cytoplasm of the mammalian cells, and the plasmid DNA is transferred to the nucleus.

Electroporation, the application of brief, high-voltage electric pulses to a variety of mammalian and plant cells leads to the formation of nanometer-sized pores in the plasma membrane. DNA is taken directly into the cell cytoplasm either through these pores or as a consequence of the redistribution of membrane components that accompanies closure of the pores. Electroporation can be extremely efficient and can be used both for transient expression of clones genes and for establishment of cell lines that carry integrated copies of the gene of interest.

Such techniques can be used for both stable and transient transformation of eukaryotic cells. The isolation of stably transformed cells requires the introduction of a selectable marker in conjunction with the transformation with the gene of interest. Such selectable markers include genes which confer resistance to neomycin as well as the HPRT gene in HPRT negative cells. Alternatively, stable transformants can be made in cells such as CHO-DG44 which are DHFR negative using DHFR containing vectors. Selection can require prolonged culture in selection media, at least for about 2-7 days, preferable for at least about 1-5 weeks (see, e.g., Sambrook & Russell, Molecular Cloning: A Laboratory Manual, Cold Spring Harbor Laboratory Press, New York (2001), pp. 16.1-16.54). Eucaryotic cell lines with high antibody productivity can be obtained using expression systems that rely upon insertion of the antibody construct into a transcriptionally active region or systems that link the antibody gene to an amplifiable gene (e.g. DHFR).

A light or heavy chain of stabilized anti-CD20 antibody polypeptide can be expressed and purified from a recombinant host cell. Recombinant host cells may be prokaryotic or eukaryotic, including but not limited to bacteria such as E. coli, fungal cells such as yeast, insect cells including but, not limited to, drosophila and silkworm derived cell lines, and mammalian cells and cell lines.

Issues which must be considered for optimal polypeptide expression in prokaryotes include the expression systems used, selection of host strain, mRNA stability, codon bias, inclusion body formation and prevention, fusion protein and site-specific proteolysis, compartment directed secretion. (See, e.g., Sorensen et al., Journal of Biotechnology 115 (2005) 113-128, which is hereby incorporated by reference).

Expression is normally induced from a plasmid harbored by a system compatible genetic background. The genetic elements of the expression plasmid include origin of replication (ori), an antibiotic resistance marker, transcriptional promoters, translation initiation regions (TIRs) as well as transcriptionil and translational terminators.

Any suitable expression system can be used, for example, E. coli facilitates protein expression by its relative simplicity, high-density cultivation, the well-known genetics and the large number of compatible tools, including a variety of available plasmids, recombinant fusion partners and mutant strains, that are available for polypeptide expression. The E. coli strain or genetic background for recombinant expression is highly important. Expression strains should be deficient in the most harmful natural proteases, maintain the expression plasmid stably and confer the genetic elements relevant to the expression system (e.g., DE3).

Plasmid copy number is controlled by the origin of replication that preferably replicates in a relaxed fashion. The ColEI replicon present in modern expression plasmids is derived from the pBR322 (copy number 15-20) or the pUC (copy number 500-700) family ofplasmids, whereas the p15A replicon is derived from pACYC184 (copy number 10-12). The most common drug resistance markers in recombinant expression plasmids confer resistance to ampicillin, kanamycin, chloramphenicol or tetracycline.

Suitable E. coli expression systems include, but are not limited to, generic pUC vectors, T7 based pET expression systems (EMD Chemicals Inc., Gibbstown, N.J.; Agilent Technologies, Inc., Wilmington, Del.), lambda PL promoter/cI repressor (e.g., pLEX (Life Technologies, Grand Island, N.Y.)), Trc promoter (e.g., pTrc (GE Healthcare Biosciences, Piscataway, N.J.)), Tac promoter (e.g., pGEX (GE Healthcare Biosciences, Piscataway, N.J.)) and hybrid lac/T5 (e.g., pQE (Qiagen, Valencia, Calif.)) and the BAD promoter (e.g., pBAD (Life Technologies, Grand Island, N.Y.)).

Translation initiation from the translation initiation region (TIR) of the transcribed messenger RNA require a ribosomal binding site (RBS) including the Shine-Dalgarno (SD) sequence and a translation initiation codon. The Shine-Dalgarno sequence is located 7±2 nucleotides upstream from the initiation codon, which is the canonical AUG in efficient recombinant expression systems. Optimal translation initiation is obtained from mRNAs with the SD sequence UAAGGAGG.

Codon usage in E. coli is reflected by the level of cognate amino-acylated tRNAs available in the cytoplasm. Major codons occur in highly expressed genes whereas the minor or rare codons tend to be in genes expressed at low levels. Codons rare in E. coli are often abundant in heterologous genes from sources such as eukaryotes, archaeabacteria and other distantly related organisms with different codon frequency preferences. Expression of genes containing rare codons can lead to translational errors, as a result of ribosomal stalling at positions requiring incorporation of amino acids coupled to minor codon tRNAs. Codon bias problems become highly prevalent in recombinant expression systems, when transcripts containing rare codons in clusters, such as doublets and triplets accumulate in large quantities. When expressing a light or heavy chain of stabilized anti-CD20 antibodies polypeptide in a nonhuman cell, whether, in vitro or in vivo, the codons selected for such the polynucleotide encoding the peptide can be optimized for a given cell type (i.e., species). Many techniques for codon optimization are known in the art (see, e.g., Jayaraj et al., Nucleic Acids Res. 33(9):3011-6 (2005); Fuglsang et al., Protein Expr. Purif. 31(2):247-9 (2003))

Protein activity demands folding into precise three dimensional structures. Stress situations such as heat shock impair folding in vivo and folding intermediates tend to associate into amorphous protein granules termed inclusion bodies.

Inclusion bodies are a set of structurally complex aggregates often perceived to occur as a stress response when recombinant protein is expressed at high rates. Macromolecular crowding of proteins at concentrations of 200-300 mg/ml in the cytoplasm of E. coli, suggest a highly unfavorable-protein-folding environment, especially during recombinant high-level expression. Whether inclusion bodies form through a passive event occurring by hydrophobic interaction between exposed patches on unfolded chains or by specific clustering mechanisms is unknown. The purified aggregates can be solubilized using detergents like urea and guadinium hydrochloride. Functional protein can be prepared by in vitro refolding from solubilized inclusion bodies either by dilution, dialysis or on-column refolding methods. Refolding strategies might be improved by inclusion of molecular chaperones. Optimization of the refolding procedure for a given protein however require time consuming efforts and is not always conducive to high product yields. A possible strategy for the prevention of inclusion body formation is the co-overexpression of molecular chaperones.

A wide range of protein fusion partners has been developed in order to simplify the purification and expression of recombinant proteins. Fusion proteins or chimeric proteins usually include a partner or “tag” linked to the passenger or target protein by a recognition site for a specific protease. Most fusion partners are exploited for specific affinity purification strategies. Fusion partners are also advantageous in vivo, where they might protect passengers from intracellular proteolysis, enhance solubility or be used as specific expression reporters. High expression levels can often be transferred from a N-terminal fusion partner, to a poorly expressing passenger, most probably as a result of mRNA stabilization. Common affinity tags are the polyhistidine tag (His-tag), which is compatible with immobilized metal affinity chromatography (IMAC) and the glutathione S-transferase (GST) tag for purification on glutathione based resins. Several other affinity tags exist and have been extensively reviewed.

Recombinantly expressed proteins can in principle be directed to three different locations namely the cytoplasm, the periplasm or the cultivation medium. Various advantages and disadvantages are related to the direction of a recombinant protein to a specific cellular compartment. Expression in the cytoplasm is normally preferable since production yields are high. Disulfide bond formation is segregated in E. coli and is actively catalyzed in the periplasm by the Dsb system. Alternatively, trxB and gor strains can be used. Reduction of cysteines in the cytoplasm is achieved by thioredoxin and glutaredoxin. Thioredoxin is kept reduced by thioredoxin reductase and glutaredoxin by glutathione. The low molecular weight glutathione molecule is reduced by glutathione reductase. Disruption of the trxB and gor genes encoding the two reductases, allow the formation of disulfide bonds in the E. coli cytoplasm.

Cell-free systems for in vitro gene expression and protein synthesis have been described for many different prokaryotic and eukaryotic systems (see, e.g., Endo & Sawasaki Current Opinion in Biotechnology 2006, 17:373-380). Eukaryotic cell-free systems, such as rabbit reticulocyte lysate and wheat germ extract, are prepared from crude extract containing all the components required for translation of in vitro-transcribed RNA templates. Eukaryotic cell-free systems use isolated RNA synthesized in vivo or in vitro as a template for the translation reaction (e.g. Rabbit Reticulocyte Lysate Systems or Wheat Germ Extract Systems). Coupled eukaryotic cell-free systems combine a prokaryotic phage RNA polymerase with eukaryotic extracts and utilize an exogenous DNA or PCR-generated templates with a phage promoter for in vitro protein synthesis (e.g., TNT® Coupled Reticulocyte Lysate

Solubility of a purified light or heavy chain of stabilized anti-CD20 antibody polypeptides can be improved by methods known in the art. For example, to increase the solubility of an expressed protein (e.g., in E. coli), one can reduce the rate of protein synthesis by lowering the growth temperature, using a weaker promoler, using a lower copy number plasmid, lowering the inducer concentration, changing the growth medium as described in Georgiou & Valax, Current Opinion Biotechnol. 7:190-197 (1996). This decreases the rate of protein synthesis and usually more soluble protein is obtained. One can also add prosthetic groups or co-factors which are essential for proper folding or for protein stability, or add buffer to control pH fluctuation in the medium during growth, or add 1% glucose to repress induction of the lac promoter by lactose, which is present in most rich media (such as LB, 2xYT). Polyols (e.g., sorbitol) and sucrose may also be added to the media because the increase in osmotic pressure caused by these additions leads to the accumulation of osmoprotectants in the cell, which stabilize the native protein structure. Ethanol, low molecular weight thiols and disulfides, and NaCl may be added. In addition, chaperones and/or foldases may be co-expressed with the desired polypeptide. Molecular chaperones promote the proper isomerization and cellular targeting by transiently interacting with folding intermediates. E. coli chaperone systems include but, are not limited to: GroES-GroEL, DnaK-DnaJ-GrpE, CIpB, FkpA, Skp. FkpA, a periplasmic peptidyl-prolyl cis,trans-isomerase, is particularly suitable.

Foldases accelerate rate-limiting steps along the folding pathway. Three types of foldases play an important role: peptidyl prolyl cis/trans isomerases (PPI's)(FkpA), disulfide oxidoreductase (DsbA) and disulfide isomerase (DsbC), protein disulfide isomerase (PDI) which is an eukaryotic protein that catalyzes both protein cysteine oxidation and disulfide bond isomerization. Co-expression of one or more of these proteins with the target protein could lead to higher levels of soluble target protein.

A light or heavy chain of stabilized anti-CD20 antibody polypeptide can be produced as a fusion protein in order to improve its solubility and production. The fusion protein comprises a light or heavy chain of stabilized anti-CD20 antibody polypeptide and a second polypeptide fused together in frame. The second polypeptide may be a fusion partner known in the art to improve the solubility of the polypeptide to which it is fused, for example, NusA, bacterioferritin (BFR), GrpE, thioredoxin (TRX), maltose binding protein (MBP) and glutathione-S-transferase. (GST). Novagen Inc. (Madison, Wis.) provides the pET 43.1 vector series which permit the formation of a NusA-target fusion. DsbA and DsbC have also shown positive effects on expression levels when used as a fusion partner, therefore can be used to fuse with a peptide ligand domain for achieving higher solubility.

In an aspect of such fusion proteins, the expressed light or heavy chain of stabilized anti-CD20 antibody polypeptide includes a linker polypeptide comprises a protease cleavage site comprising a peptide bond which is hydrolyzable by a protease. As a result, the peptide ligand domain in a polypeptide can be separated from the remainder of the polypeptide after expression by proteolysis. The linker can comprise one or more additional amino acids on either side of the bond to which the catalytic site of the protease also binds (see, e.g., Schecter & Berger, Biochem. Biophys. Res. Commun. 27, 157-62 (1967)). Alternatively, the cleavage site of the linker can be separate from the recognition site of the protease and the two cleavage site and recognition site can be separated by one or more (e.g., two to four) amino acids. In one aspect, the linker comprises at least about 2, 3, 4, 5, 6, 7, 8, 9, about 10, about 20, about 30, about 40, about 50 or more amino acids. More preferably the linker is from about 5 to about 25 amino acids in length, and most preferably, the linker is from about 8 to about 15 amino acids in length.

For example, suitable proteases that are commonly used with bacterially-produced fusion proteins include tobacco etch virus (TEV) protease, factor Xa protease, thrombin and enterokinase. Some proteases useful with the invention are discussed in the following references: Hooper et al., Biochem. J. 321: 265-279 (1997); Werb, Cell 91: 439-442 (1997); Wolfsberg et al., J. Cell Biol. 131: 275-278 (1995); Murakami & Etlinger, Biochem. Biophys. Res. Comm. 146: 1249-1259 (1987). Cell surface proteases also can be used with cleavable linkers according to the invention and include, but are not limited to: Aminopeptidase N; Puromycin sensitive aminopeptidase; Angiotensin converting enzyme; Pyroglutamyl peptidase II; Dipeptidyl peptidase IV; N-arginine dibasic convertase; Endopeptidase 24.15; Endopeptidase 24.16; Amyloid precursor protein secretases alpha, beta and gamma; Angiotensin converting enzyme secretase; TGF alpha secretase; TNF alpha secretase; FAS ligand secretase; TNF receptor-I and -II secretases; CD30 secretase; KL1 and KL2 secretases; IL6 receptor secretase; CD43, CD44 secretase; CD16-I and CD16-II secretases; L-selectin secretase; Folate receptor secretase; MMP 1, 2, 3, 7, 8, 9, 10, 11, 12, 13, 14, and 15; Urokinase plasminogen activator; Tissue plasmninogen activator; Plasmin; Thrombin; BMP-1 (procollagen C-peptidase); ADAM 1, 2, 3, 4, 5, 6, 7, 8, 9, 10, and 1); and, Granzymnes A, B, C, D, E, F, G, and H.

An alternative to relying on cell-associated proteases is to use a self-cleaving linker. For example, the foot and mouth disease virus (FMDV) 2A protease may be used as a linker. This is a short polypeptide of 17 amino acids that cleaves the polyprotein of FMDV at the 2A/2B junction. The sequence of the FMDV 2A propeptide is NFDLLKLAGDVESNPGP. Cleavage occurs at the C-terminus of the peptide at the final glycine-proline amino acid pair and is independent of the presence of other FMDV sequences and cleaves even in the presence of heterologous sequences.

Affinity chromatography can be used alone or in conjunction with ion-exchange, molecular sizing, or HPLC chromatographic techniques in the purification of the polypeptides making up the light and heavy chains of stabilized anti-CD20 antibodies. Such chromatographic approach can be performed using columns or in batch formats. Such chromatographic purification methods are well known in the art.

The Large Scale Production of Anti-CD20 Antibodies

There two major types of recombinant expressions of biological products, one is the soluble form of biological product that is secreted into nutrient medium by the cells as most often seen in the use of Chinese Hamster Ovary cells and the other is the retention of biological product inside the cell forming an inclusion body, as most often seen in the case of using E. coli for expression. Recent advances in genetic engineering have been able to encode the genes of bacteria that would secrete soluble proteins instead of retain them inside as inclusion bodies. This is to avoid the cumbersome process of cell-lysis and inclusion body solubilization.

Any suitable method can be used to produce anti-CD20 antibodies on a large scale. For example, a variety of vessels and methods have been developed over the years to carry out the fermentation of microorganisms, particularly bacteria and yeast, on a commercial scale. Stainless steel fermentation vessels of several hundreds of thousands liters are not uncommon, with the fermentation methods including batch, fed-batch, continuous or semi-continuous perfusion. The cells within these vessels are desirably kept in suspension, typically by rotating stirring blades located within the vessel, with gas exchange facilitated by the injection of air, oxygen or carbon dioxide into the vessel.

In addition, disposable fermentors are also known to those of ordinary skill. Examples of such disposable fermentors are systems based on wave agitation. (See, e.g., U.S. Pat. No. 6,544,788; PCT Publication WO 00/66706.) This type of fermentor can be used to culture relatively sensitive cells such as CHO cells (e.g. Pierce, Bioprocessing J. 3: 51-56 (2004)), hybridoma cells (e.g., Ling et al., Biotech. Prog., 19: 158-162 (2003)), insect cells (e.g., Weber et al., Cytotech. 38: 77-85 (2002)) and anchorage-dependent cells (e.g., Singh, Cytotech. 30: 149-158 (1999)) in a single disposable container. Such disposable units are relatively cheap, decrease the risk of infection because of their single use and require no internal stirring parts as the rocking platform upon which these containers reside during use induces wave-like forms in the internal liquid which facilitates gas exchange.

Another suitable method provides in one aspect a bioreactor for use in preparing a variety of biological products (see, e.g., U.S. Pat. Appl. Pub. No. 20110117538.) This bioreactor is suitable for housing a predetermined volume of liquid comprising nutrient medium and biological culture and comprises: (a) a container having at least one interior wall; (b) at least one inlet; (c) at least one outlet; (d) at least one gas inlet; (e) at least one gas outlet; and (ft) at least one cylindrical sparging filter attached to the at least one gas inlet, wherein the sparging filter comprises a plurality of pores along its axis which permit gas to be emitted radially from the sparging filter into the liquid, wherein the diameter of the plurality of pores does not exceed about 50 μm, and wherein the orientation of the at least one sparging filter within the container provides for immersion of the plurality of pores within the liquid and substantially uniform distribution of emitted gas throughout the liquid.

A related aspect of such bioreactor systems disclosed in U.S. Pat. Appl. Pub. No. 20110117538, which provides a method for producing a biological product from a predetermined volume of a liquid comprising nutrient medium and biological culture comprising (a) providing a bioreactor; (b) introducing nutrient medium and biological culture into the container; (c) passing gas through a sparging filter and into the liquid; (d) detecting the density of cells in the liquid at predetermined time intervals; and (e) removing the liquid and any biological product produced thereby from the container when the density of the cells in the liquid within the container reaches a predetermined value.

Another suitable method provides a bioreactor such as the one disclosed in U.S. Pat. Appl. Pub. No. 20110198286. This system capitalizes on the recent availability of many resins that are capable of binding biological products in large quantities. Most modern resins bind between 20-125 mg of biological product per mL of resin. Many of these resins are highly specific to the biological products and many of them can be combined to remove any type and quantity of a biological product from a solution by a simple process of physicochemical binding that is strong enough to retain the biological products attached to the resin while the culture medium is removed from the bioreactor. The field of biological product purification wherein now has the ability to elute these bound biological products from resins by adjusting the pH, the ionic strength or other characteristics of the eluting buffer to break the binding between the resin and the biological product. This allows removal of biological products from a bioreactor as a highly concentrated solution that is ready for further purification and in some instances it can even be the final product for use.

Coupled Anti-CD20 Antibody Compositions

The invention provides compositions comprising the inventive anti-CD20 antibodies disclosed herein. In preferred embodiments, the composition is a pharmaceutically acceptable composition comprising an anti-CD20 antibody and a pharmaceutically acceptable carrier.

Antibodies can act to remove cancerous cells through several effector mechanisms. For antibodies that are fully human or chimeric, the Fc portion of the molecule can efficiently activate and interact with the human immune system. By this method, cells can be destroyed by soluble components of the immune system (complement) or through ADCC-mediated cell killing. Additionally, binding of the antibody to the target antigen can initiate a biological response that can lead to apoptosis. Further, antibody molecules can be used as delivery vehicles to transport therapeutic moieties such as drugs, radioisotopes, toxins, or enzymes.

The compositions of the present invention can further comprise an active agent. In some embodiments, the active agent is a pharmaceutically active therapeutic agent or “effector” directly able to exert its pharmacological effect. In other embodiments, the active agent is a diagnostic agent. It will be understood that some active agents are useful as both diagnostic and therapeutic agents, and therefore such terms are not mutually exclusive. In preferred embodiments, the active agent is a diagnostic or therapeutic active agent conjugated or coupled to an anti-CD20 antibody.

Methods for coupling or conjugation of suitable therapeutics, chemotherapeutics, radionuclides, etc. to antibodies or fragments thereof are well described in the art. For example, without limitation, free amino groups in proteins, such the epsilon-amino group of lysine, can be conjugated with reagents such as carbodiimides or heterobiofunctional agents. Alternatively, e.g., sulfhydryl groups can be used for conjugation. In addition, sugar moieties bound to glycoproteins, including antibodies, can be oxidized to form aldehydes groups useful in a number of coupling procedures known in the art. The conjugates formed in accordance with the invention can be stable in vivo or labile, such as enzymatically degradeable tetrapeptide linkages or acid-labile cis-aconityl or hydrazone linkages. In addition, the invention provides for anti-CD20 antibody fusion proteins, including, for example without limitation, wherein heavy or light chain encoding sequences (or encoding fragments thereof, such as Fab, Fv, Fab′ and Fab′2 fragments) are fused upstream or downstream of diagnostically useful protein domains (such as hapten, GFP), immunologically active protein domains (e.g., TF or TNF) or toxin domains.

Compositions of the present invention can be used to enhance delivery of the active agent to a disease site relative to delivery of the active agent alone. In preferred embodiment coupling to the anti-CD20 antibody increases the level of the active at the disease site by at least 10%, at least 20%, at least 25%, at least 50%, at least 100%, at least 3 fold, at least 5 fold, at least 10 fold, at least 20 fold, at least 50 fold, at least 100 fold, in comparison to the level achieved at the disease site by uncoupled active.

As used herein, the phrases “indirect labeling” and “indirect labeling approach” both mean that a chelating agent is covalently attached to an antibody and at least one radionuclide is inserted into the chelating agent. Suitable chelating agents and radionuclides are set forth in Srivagtava, S. C. and Mease, R. C.,” Progress in Research on Ligands, Nuclides and Techniques for Labeling Monoclonal Antibodies,” Nucl. Med. Bio. 18/6: 589-603 (1991) which is incorporated herein by reference. A particularly preferred chelating agent is 1-isothiocycmatobenzyl-3-methyldiothelene triaminepent acetic acid (“MX-DTPA”); particularly preferred radionuclides for indirect labeling include indium-111 and yttrium-90. As used herein, the phrases “direct labeling” and “direct labeling approach” both mean that a radionuclide is covalently attached directly to an antibody (typically via an amino acid residue). Suitable radionuclides are provided in Srivagtava; or cite a particularly preferred radionuclide for direct labeling is iodine-131 covalently attached via tyrosine residues. The indirect labeling approach is particularly preferred.

The agent used for coupling to the anti-CD20 antibody can be any suitable therapeutic agent (or “effector”) or diagnostic agent, such as a chemotherapeutic or anticancer agent. Suitable diagnostic agents include fluorochromes, radioactive agents, MRI contrast agents, X-ray contrast agents, ultrasound contrast agents, and PET contrast agents. Suitable chemotherapeutic agents or other anticancer agents for use in accordance with the invention include, but are not limited to, tyrosine kinase inhibitors (genistein), biologically active agents (TNF, tTF), radionuclides (¹³¹I, ⁹⁰Y, ¹¹¹In, ²¹¹At, ³²P and other known therapeutic radionuclides), adriamycin, ansamycin antibiotics, asparaginase, bleomycin, busulphan, cisplatin, carboplatin, carmustine, capecitabine, chlorambucil, cytarabine, cyclophosphamide, camptothecin, dacarbazine, dactinomycin, daunorubicin, dexrazoxane, docetaxcel, doxorubicin, etoposide, epothilones, floxuridine, fludarabine, fluorouracil, gemcitabine, hydroxyurea, idarubicin, ifosfamide, irinotecan, lomustine, mechlorethamine, mercaptopurine, meplhalan, methotrexate, rapamycin (sirolimus) and derivatives, mitomycin, mitotane, mitoxantrone, nitrosurea, paclitaxel, pamidronate, pentostatin, plicamycin, procarbazine, rituximab, streptozocin, teniposide, thioguanine, thiotepa, taxanes, vinblastine, vincristine, vinorelbine, taxol, combretastatins, discodermolides, and transplatinum.

Other suitable chemotherapeutic agents for use in accordance with invention include, without limitation, antimetabolites (e.g., asparaginase), antimitotics (e.g., vinca alkaloids), DNA damaging agents (e.g., cisplatin), proapoptotics (agents which induce programmed-cell-death or apoptosis) (e.g., epipodophylotoxins), differentiation inducing agents (e.g., retinoids), antibiotics (e.g., bleomycin), and hormones (e.g., tamoxifen, diethylstibestrol). Further, suitable chemotherapeutic agents for use in accordance with the invention include antiangiogenesis agents (angiogenesis inhibitors) such as, e.g., IFN-alpha, fumagillin, angiostatin, endostatin, thalidomide, and the like.

In addition, the pharmaceutically active agent can be an siRNA. In preferred embodiments, the siRNA molecule inhibits expression of an gene associated with tumors such as, for example, c-Sis and other growth factors, EGFR, PDGFR, VEGFR, HER2, other receptor tyrosine kinases, Src-family genes, Syk-ZAP-70 family genes, BTK family genes, other cytoplasmic tyrosine kinases, Raf kinase, cyclin dependent kinases, other cytoplasmic serine/threonine kinases, Ras protein and other regulatory GTPases.

Anti-CD20 antibodies can also be conjugated to polyethylene glycol (PEG). PEG conjugation can increase the circulating half-life of a protein, reduce the protein's immunogenicity and antigenicity, and improve the bioactivity. Any suitable method of conjugation can be used, including but not limited to, e.g., reacting methoxy-PEG with a CD20 binding antibody's available amino groups or other reactive sites such as, e.g., histidines or cysteines. In addition, recombinant DNA approaches can be used to add amino acids with PEG-reactive groups to the inventive CD20 binding antibodies. PEG can be processed prior to reacting it with a CD20 binding antibody, e.g., linker groups can be added to the PEG. Further, releasable and hybrid PEG-ylation strategies can be used in accordance with the invention, such as, e.g., the PEG-ylation of a CD20 binding antibody such that the PEG molecules added to certain sites in the CD20 binding antibody are released in vivo. Such PEG conjugation methods are known in the art (see, e.g., Greenwald et al., Adv. Drug Delivery Rev. 55:217-250 (2003)).

Anti-CD20 Antibody Formulations

The compositions of the present inventions are generally provided in a formulation with a carrier, such as a pharmaceutically acceptable carrier. Typically, the carrier will be liquid, but also can be solid, or a combination of liquid and solid components. The carrier desirably is a physiologically acceptable (e.g., a pharmaceutically or pharmacologically acceptable) carrier (e.g., excipient or diluent). Suitable pharmaceutical excipients include stabilizers, antioxidants, osmolality adjusting agents, buffers, and pH adjusting agents. Suitable additives include physiologically biocompatible buffers, additions of chelants or calcium chelate complexes, or, optionally, additions of calcium or sodium salts. Pharmaceutical compositions can be packaged for use in liquid form, or can be lyophilized. Preferred physiologically acceptable carrier media are water, buffered water, normal saline, 0.4% saline, 0.3% glycine, hyaluronic acid and the like. Physiologically acceptable carriers are well known and are readily available. The choice of carrier will be determined, at least inl part, by the location of the target tissue and/or cells, and the particular method used to administer the composition.

The composition can be formulated for administration by a route including intravenous, intraarterial, intramuscular, intraperitoneal, intrathecal, epidural, of subcutaneous, transmucosal (including, for example, pulmonary). The composition also can comprise additional components such as diluents, adjuvants, excipients, preservatives, and pH adjusting agents, and the like.

Formulations suitable for injectable administration include aqueous and nonaqueous, isotonic sterile injection solutions, which can contain anti-oxidants, buffers, bacteriostats, and solutes that render the formulation isotonic with the blood of the intended recipient, and aqueous and nonaqueous sterile suspensions that can include suspending agents, solubilizers, thickening agents, stabilizers, lyoprotectants, and preservatives. The formulations can be presented in unit-dose or multi-dose sealed containers, such as ampules and vials, and can be stored in a freeze-dried (lyophilized) condition requiring only the addition of the sterile liquid carrier, for example, water, for injections, immediately prior to use. Extemporaneous injection solutions and suspensions can be prepared from sterile powders, granules, or tablets.

Sterile injectable solutions can be prepared by incorporating the active compound in the required amount in an appropriate solvent with one or a combination of ingredients enumerated above, as required, followed by filtered sterilization. Preferably solutions for injection are free of endotoxin. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof. In all cases, the formulation must be sterile and must be fluid to the extent that easy syringability exists. It must be stable under the conditions of manufacture and storage and must be preserved against the contaminating action of microorganisms, such as bacteria and fungi. Solutions of the active compounds as free base or pharmacologically acceptable salts can be prepared in water suitably mixed with a surfactant, such as hydroxycellulose. Dispersions can also be prepared in glycerol, liquid polyethylene glycols, and mixtures thereof and in oils. Under ordinary conditions of storage and use, these preparations contain a preservative to prevent the growth of microorganisms.

In preferred embodiments, the active ingredients can be entrapped in microcapsules prepared, for example, by coacervation techniques or by interfacial polymerization, for example, hydroxymethylcellulose or gelatin-microcapsule and poly-(methylmethacylate) microcapsule, respectively, in colloidal drug delivery systems (for example, liposomes, albumin microspheres, microemulsions, nano-particles and nanocapsules) or in macroemulsions. Suitable techniques are disclosed in Rezler et al., J. Am. Chem. Soc. 129(16): 4961-72 (2007); Samad et al., Curr. Drug Deliv. 4(4): 297-305 (2007); and U.S. Pat. Nos. 4,485,045 and 4,544,545. Liposomes with enhanced circulation time are disclosed in U.S. Pat. No. 5,013,556.

Particularly useful liposomes can be generated by, for example, the reverse-phase evaporation method with a lipid composition comprising phosphatidylcholine, cholesterol and PEG-derivatized phosphatidylethanolamine (PEG-PE).

The pharmaceutical compositions can be delivered using drug delivery systems. Such delivery systems include hyaluronic acid solutions or suspensions of collagen fragments. The drugs can be formulated in microcapsules, designed with appropriate polymeric materials for controlled release, such as polylactic acid, ethylhydroxycellulose, polycaprolactone, polycaprolactone diol, polylysine, polyglycolic, polymaleic acid, poly[N-(2-hydroxypropyl)methylacrylamide] and the like. Particular formulations using drug delivery systems can be in the form of liquid suspensions, ointments, complexes to a bandage, collagen shield or the like.

The composition can further comprise any other suitable components, especially for enhancing the stability of the composition and/or its end-use. Accordingly, there is a wide variety of suitable formulations of the composition of the invention.

Compositions provided by the invention can include, e.g., from about 0.5 mL to about 4 mL aqueous or organic liquids with an active agent coupled to a CD20 binding antibody, with the concentration of the active agent from about 10 mg/mL to about 100 mg/mL, preferably from about 1 mg/mL to about 10 mg/mL, more preferably from about 0.1 mg/mL to about 1 mg/mL.

Methods of Treatment and Diagnosis with anti-CD20 Antibodies

The invention provides a method for diagnosing or treating a disease in an animal by administering a diagnostically or therapeutically effective amount of a composition comprising an anti-CD20 antibody. In particular, the invention provides methods of treating a disease in a patient comprising administering to the animal a therapeutically effective amount of an anti-CD20 antibody

Preferred effective dosages (i.e., therapeutically effective amounts) of the immunologically active chimeric anti-CD20 antibodies range from about 0.001 to about 30 mg/kg body weight, more preferably from about 0.01 to about 25 mg/kg body weight, and even more preferably from about 0.4 to about 20.0 mg/kg body weight. Alternatively, the preferred effective dosages may be described as from about 250 mg/m² to about 500 mg/m², more preferably as about 375 mg/m².

However, other dosages are viable; factors influencing dosage include, but are not limited to, the severity of the disease; previous treatment approaches; overall health of the patient; other diseases present, and the like. The skilled artisan is readily able to assessing a particular patient and determining a suitable dosage that falls within the ranges, or if necessary, outside of the ranges.

Accordingly, any suitable dosage level for anti-CD20 antibodies can be used in accordance with the invention, e.g., dosage levels on the order of about 1 μg/kg to 100 mg/kg of body weight per administration are useful in the treatment of a disease. With regard to suitable dosages, an antibody can be administered at a unit dose less than about 75 mg per kg of bodyweight, or less than about 70, 60, 50, 40, 30, 20, 10, 5, 2, 1, 0.5, 0.1, 0.05, 0.01, 0.005, 0.001, or 0.0005 mg per kg of bodyweight, and less than 200 nmol of antibody per kg of bodyweight, or less than 1500, 750, 300, 150, 75, 15, 7.5, 1.5, 0.75, 0.15, 0.075, 0.015, 0.0075, 0.0015, 0.00075, 0.00015 nmol of antibody per kg of bodyweight. The unit close, for example, can be administered by injection (e.g., intravenous or intramuscular, intrathecally, or directly into an organ), inhalation, or a topical application.

Likewise, the invention further provides methods of treating a tumor in an animal with one or more anticancer agents and an anti-CD20 antibody comprising: isolating a biological sample (spccimen) from the animal, detecting the expression of CD20 protein or RNAs in the biological sample, or quantifying the amount of CD20 protein or RNAs in the biological sample, and if the CD20 protein or RNA in the biological sample is present above a threshold level administering a therapeutically effective amount of the anticancer agent and a therapeutically effective amount of the anti-CD20 antibody.

The level of CD20 protein present in a sample is typically detected using an anti-CD20 antibody in a blot or ELISA assay. However, in some embodiments, the expression of a CD20 protein can be determined using only a portion of an antibody, using a CD20 binding molecule which is not an antibody, or using some other method of detecting CD20 expression not requiring an antibody or a CD20 binding molecule, e.g., mass spectroscopy. The CD20 RNA level may be obtained by any suitable method, including e.g., Northern blot, slot blot, microarray analysis, quantitative PCR, quantitative TMA, and quantitative invader.

The invention also provides a method for inhibition of CD20 activity using a suitable neutralizing anti-CD20 antibody. Such a neutralizing antibody can, e.g., have the ability to block the interaction of anti-CD20 with a soluable or cell surface ligand or prevent CD20 confirmational changes required for signal transduction.

In other embodiments, the inventive methods comprise administering to a mammal a therapeutically effective amount of a pharmaceutical composition comprising a liposome bound or albumin bound chemotherapeutic agent wherein the liposome or albumin is coupled to a suitable disease targeting anti-CD20 antibody. The chemotherapeutic agent can be coupled to the anti-CD20 antibody using any suitable method. Preferably, the chemotherapeutic agent is chemically coupled to the compound via covalent bonds including, for example, disulfide bonds.

One or more doses of one or more chemotherapeutic agents, such as those described above, can also be administered according to the inventive methods. The type and number of chemotherapeutic agents used in the inventive method will depend on the standard chemotherapeutic regimen for a particular tumor type. In other words, while a particular cancer can be treated routinely with a single chemotherapeutic agent, another can be treated routinely with a combination of chemotherapeutic agents. The following examples further illustrate the invention but, of course, should not be construed as in any way limiting its scope.

Methods in accordance with the invention include, e.g., combination therapies wherein the animal is also undergoing one or more cancer therapies selected from the group consisting of surgery, chemotherapy, radiotherapy, thermotherapy, immunotherapy, hormone therapy and laser therapy. The terms “co-administration” and “combination therapy” refer to administering to a subject two or more therapeutically active agents. The agents can be contained in a single pharmaceutical composition and be administered at the same time, or the agents can be contained in separate formulation and administered serially to a subject. So long as the two agents can be detected in the subject at the same time, the two agents are said to be co-administered.

Combination therapies contemplated in the present invention include, but are not limited to, concaminant antibody administration, vaccine administration, administration of cytotoxic agents, natural amino acid polypeptides, nucleic acids, nucleotide analogues, and biologic response modifiers. Two or more combined compounds may be used together or sequentially. Examples of chemotherapeutic agents include alkylating agents, antimetabolites, natural products, hormones and antagonists, and miscellaneous agents. Examples of alkylating agents include nitrogen mustards such as mechlorethamine, cyclophosphamide, ifosfamide, melphalan (L-sarcolysin) and chlorambucil; ethylenimines and methylmelamines such as hexamethylmelamine and thiotepa; alkyl sulfonates such as busulfan; nitrosoureas such as carmustine (BCNU), semustine (methyl-CCNU), lomustine (CCNU) and streptozocin (streptozotocin); DNA synthesis antagonists such as estramustine phosphate; and triazines such as dacarbazine (DTIC, dimethyl-triazenoimidazolecarboxamide) and temozolomide. Examples of antimetabolites include folic acid analogs such as methotrexate (amethopterin); pyrimidine analogs such as fluorouracin (5-fluorouracil, 5-FU. 5FU), floxuridine (fluorodeoxyuridine, FUdR), cytarabine (cytosine arabinoside) and gemcitabine; purine analogs such as mercaptopurine (6-niercaptopurine, 6-MP), thioguanine (6-thioguanine, TG) and pentostatin (2′-deoxycoformycin, deoxycoformycin), cladribine and fludarabine; and topoisomerase inhibitors, such as amsacrine. Examples of natural products include vinca alkaloids such as vinblastine (VLB) and vincristine; taxanes such as paclitaxel (Abraxane®) and docetaxel (Taxotere®); epipodophyllotoxins such as etoposide and teniposide; camptothecins such as topotecan and irinotecan; antibiotics such as dactinomycin (actinomycin D), daunorubicin (daunomycin, rubidomycin), doxorubicin, bleomycin, mitomycin (mitomycin C), idarubicin, epirubicin; enzymes such as L-asparaginase; and biological response modifiers such as interferon alpha and interleukin 2. Examples of hormones and antagonists include luteinising releasing hormone agonists such as buserelin; adrenocorticosteroids such as prednisone and related preparations; progestins such as hydroxyprogesterone caproate, medroxyprogesterone acetate and megestrol acetate; estrogens such as diethylstilbestrol and ethinyl estradiol and related preparations; estrogen antagonists such as tamoxifen and anastrozole; androgens such as testosterone propionate and fluoxymesterone and related preparations; androgen antagonists such as flutamide and bicalutamide; and gonadotropin-releasing hormone analogs such as leuprolide. Examples of miscellaneous agents include thalidomide; platinum coordination complexes such as cisplatin (czs-DDP), oxaliplatin and carboplatin; anthracenediones such as mitoxantrone; substituted ureas such as hydroxyurea; methylhydrazine derivatives such as procarbazine (N-methylhydrazine, MIH); adrenocortical suppressants such as mitotane (o,p′-DDD) and aminoglutethimide; RXR agonists such as bexarotene; and tyrosine kinase inhibitors such as imatinib.

Compositions featured in the methods of the present invention can be administered in a single dose or in multiple doses. Where the administration of the antibodies by infusion, the infusion can be a single sustained dose or can be delivered by multiple infusions. Injection of the agent can be directly into the tissue at or near the site of aberrant target gene expression. Multiple injections of the agent can be made into the tissue at or near the site.

One skilled in the art can also readily determine an appropriate dosage regimen for administering the antibody of the invention to a given subject. For example, the anti-CD20 antibody composition can be administered to the subject once, as a single injection or deposition at or near the site of CD20 expression. Compositions of the present invention can be administered daily, semi-weekly, weekly, bi-weekly, semi-monthly, monthly, bi-monthly, or at the discretion of the clinician. In some embodiments, the compositions are administered once or twice daily to a subject for a period of from about three to about twenty-eight days, more preferably from about seven to about ten days. In further embodiments, the unit dose is administered less frequently than once a day, e.g., less than every 2, 4, 8 or 30 days. In other embodiments, the unit dose is not administered with a frequency (e.g., not a regular frequency).

Where a dosage regimen comprises multiple administrations, it is understood that the effective of anti-CD20 antibody composition administered to the subject can include the total amount of antibody administered over the entire dosage regimen. One skilled in the art will appreciate that the exact individual dosages may be adjusted somewhat depending on a variety of factors, including the specific anti-CD20 antibody composition being administered, the time of administration, the route of administration, the nature of the formulation, the rate of excretion, the particular disorder being treated, the severity of the disorder, the pharmacodynamics of the oligonucleotide agent, and the age, sex, weight, and general health of the patient. Wide variations in the necessary dosage level are to be expected in view of the differing efficiencies of the various routes of administration.

The effective dose can be administered in a single dose or in two or more doses, as desired or considered appropriate under the specific circumstances. If desired to facilitate repeated or frequent infusions, implantation of a delivery device, e.g., a pump, semi-permanent stent (e.g., intravenous, intraperitoneal, intracisternal or intracapsular), or reservoir may be advisable. Following successful treatment, it may be desirable to have the patient undergo maintenance therapy to prevent the recurrence of the disease state. The concentration of the antibody composition is an amount sufficient to be effective in treating or preventing a disorder or to regulate a physiological condition in humans. The concentration or amount of antibody administered will depend on the parameters determined for the agent and the method of administration.

Certain factors may influence the dosage required to effectively treat a subject, including but not limited to the severity of the disease or disorder, previous treatments, the general health and/or age of the subject, and other diseases present. It will also be appreciated that the effective dosage of the antibody used for treatment may increase or decrease over the course of a particular treatment. Changes in dosage may result and become apparent from the results of diagnostic assays. For example, the subject can be monitored after administering an antibody composition. Based on information from the monitoring, an additional amount of the antibody composition can be administered. Persons of ordinary skill can easily determine optimum dosages, dosing methodologies and repetition rates.

The following examples further illustrate the invention but, of course, should not be construed as in any way limiting its scope.

### Example 1

This example demonstrates that a series of variant anti-CD20 antibody light and heavy chains with changed amino acid sequences can made by site-directed mutagenesis.

Site-specific mutations of cloned anti-CD20 light and heavy chain coding sequences was done using a variety of protocols well known to those of ordinary skill in the art, including the MORPH Site-Specific Plasmid DNA Mutagenesis Kit (5 Prime to 3 Prime, Inc.), QuikChange Site-Directed Mutagenesis Kit, QuikChange Multi Site-Directed Mutagenesis Kit, and QuikChange Lightning Multi Site-Directed Mutagenesis Kit (all from Agilent Technologies, Santa Clara, Calif.). Specifically, the QuikChange method used a complementary mismatched primer pair and primer extension using a non-strand displacing polymerase (Pfu). Prior to transformation, the parental template (prepared from methylating E. coli strain) was digested with DpnI (specific for methylated DNA, and does not digest the newly synthesized duplex DNA). All mutations were verified by dideoxy sequencing. Expression and purification of the resulting light and heavy chain polypeptides was done as described (Wilkins Stevens et al.: Recombinant immunoglobulin variable domains generated from synthetic genes provide a system for in vitro characterization of light chain amyloid proteins, Protein Sci. 4:421-432 (1995)). Antibody V_{H} and V_{L} domains were expressed in the E. coli host strain BL26. Small scale (30 mL) shake cultures were used to produce sufficient protein to evaluate thermal stability. The His-tagged proteins were isolated from the E. coli periplasmic fraction by incubation with Ni-NTA Agarose (Qiagen) or Ni Sepharose (GE Healthcare, Piscataway, N.J.). The affinity resin was collected on 96-well filter plates and washed. Protein was eluted with buffer containing 500 mM imidazole.

Tables 1-3 present the wild type anti-CD20 antibody light and heavy chains, respectively, whose residues are numbered both as defined according to Kabat E. A., Wu T. T., Perry H. M., Gottesman K. S. & Foeller C. (1991) In: Sequences of Proteins of Immunological Interest (ed. Kabat), Vol. 1, 5th edn. US Department of Health and Human Services, Bethesda, Md. (1991) (V_{L} and V_{H} sequences) or EU Numbering (C12 sequence) and with reference to SEQ ID NO:s 1 and 2. (Unless otherwise specified, all amino acid sequence numbering used herein is in reference to/based upon SEQ ID NOs: 1 and 2.)

TABLE 1
Wild Type Anti-CD20 Light Chain Variable Domain
Amino Acid Sequence Numbering System

| Wild Type | Wild Type | |
|---|---|---|
| Residue Number - | Residue - Number | Residue in Wild |
| Kabat Number | in SEQ ID NO: 1 | Type Sequence |
| L1 | 1 | Q |
| L2 | 2 | I |
| L3 | 3 | V |
| L4 | 4 | L |
| L5 | 5 | S |
| L6 | 6 | Q |
| L7 | 7 | S |
| L8 | 8 | P |
| L9 | 9 | A |
| L10 | 10 | I |
| L11 | 11 | L |
| L12 | 12 | S |
| L13 | 13 | A |
| L14 | 14 | S |
| L15 | 15 | P |
| L16 | 16 | G |
| L17 | 17 | E |
| L18 | 18 | K |
| L19 | 19 | V |
| L20 | 20 | T |
| L21 | 21 | M |
| L22 | 22 | T |
| L23 | 23 | C |
| L24 | 24 | R |
| L25 | 25 | A |
| L26 | 26 | S |
| L27 | 27 | S |
| L28 | — | — |
| L29 | 28 | S |
| L30 | 29 | V |
| L31 | 30 | S |
| L32 | 31 | Y |
| L33 | 32 | I |
| L34 | 33 | H |
| L35 | 34 | W |
| L36 | 35 | F |
| L37 | 36 | Q |
| L38 | 37 | Q |
| L39 | 38 | K |
| L40 | 39 | P |
| L41 | 40 | G |
| L42 | 41 | S |
| L43 | 42 | S |
| L44 | 43 | P |
| L45 | 44 | K |
| L46 | 45 | P |
| L47 | 46 | W |
| L48 | 47 | I |
| L49 | 48 | Y |
| L50 | 49 | A |
| L51 | 50 | T |
| L52 | 51 | S |
| L53 | 52 | N |
| L54 | 53 | L |
| L55 | 54 | A |
| L56 | 55 | S |
| L57 | 56 | G |
| L58 | 57 | V |
| L59 | 58 | P |
| L60 | 59 | V |
| L61 | 60 | R |
| L62 | 61 | F |
| L63 | 62 | S |
| L64 | 63 | G |
| L65 | 64 | S |
| L66 | 65 | G |
| L67 | 66 | S |
| L68 | 67 | G |
| L69 | 68 | T |
| L70 | 69 | S |
| L71 | 70 | Y |
| L72 | 71 | S |
| L73 | 72 | L |
| L74 | 73 | T |
| L75 | 74 | I |
| L76 | 75 | S |
| L77 | 76 | R |
| L78 | 77 | V |
| L79 | 78 | E |
| L80 | 79 | A |
| L81 | 80 | E |
| L82 | 81 | D |
| L83 | 82 | A |
| L84 | 83 | A |
| L85 | 84 | T |
| L86 | 85 | Y |
| L87 | 86 | Y |
| L88 | 87 | C |
| L89 | 88 | Q |
| L90 | 89 | Q |
| L91 | 90 | W |
| L92 | 91 | T |
| L93 | 92 | S |
| L94 | 93 | N |
| L95 | 94 | P |
| L96 | 95 | P |
| L97 | 96 | T |
| L98 | 97 | F |
| L99 | 98 | G |
| L100 | 99 | G |
| L101 | 100 | G |
| L102 | 101 | T |
| L103 | 102 | K |
| L104 | 103 | L |
| L105 | 104 | E |
| L106 | 105 | I |
| L107 | 106 | K |
| L108 | 107 | R |

TABLE 2
Wild Type Anti-CD20 Heavy Chain Variable Domain
Amino Acid Sequence Numbering System

| Wild Type | Wild Type | |
|---|---|---|
| Residue Number - | Residue - Number | Residue in Wild |
| Kabat Number | in SEQ ID NO: 2 | Type Sequence |
| H1 | 1 | Q |
| H2 | 2 | V |
| H3 | 3 | Q |
| H4 | 4 | L |
| H5 | 5 | Q |
| H6 | 6 | Q |
| H7 | 7 | P |
| H8 | 8 | G |
| H9 | 9 | A |
| H10 | 10 | E |
| H11 | 11 | L |
| H12 | 12 | V |
| H13 | 13 | K |
| H14 | 14 | P |
| H15 | 15 | G |
| H16 | 16 | A |
| H17 | 17 | S |
| H18 | 18 | V |
| H19 | 19 | K |
| H20 | 20 | M |
| H21 | 21 | S |
| H22 | 22 | C |
| H23 | 23 | K |
| H24 | 24 | A |
| H25 | 25 | S |
| H26 | 26 | G |
| H27 | 27 | Y |
| H28 | 28 | T |
| H29 | 29 | F |
| H30 | 30 | T |
| H31 | 31 | S |
| H32 | 32 | Y |
| H33 | 33 | N |
| H34 | 34 | M |
| H35 | 35 | H |
| H36 | 36 | W |
| H37 | 37 | V |
| H38 | 38 | K |
| H39 | 39 | Q |
| H40 | 40 | T |
| H41 | 41 | P |
| H42 | 42 | G |
| H43 | 43 | R |
| H44 | 44 | G |
| H45 | 45 | L |
| H46 | 46 | E |
| H47 | 47 | W |
| H48 | 48 | I |
| H49 | 49 | G |
| H50 | 50 | A |
| H51 | 51 | I |
| H52 | 52 | Y |
| H52A | 53 | P |
| H53 | 54 | G |
| H54 | 55 | N |
| H55 | 56 | G |
| H56 | 57 | D |
| H57 | 58 | T |
| H58 | 59 | S |
| H59 | 60 | Y |
| H60 | 61 | N |
| H61 | 62 | Q |
| H62 | 63 | K |
| H63 | 64 | F |
| H64 | 65 | K |
| H65 | 66 | G |
| H66 | 67 | K |
| H67 | 68 | A |
| H68 | 69 | T |
| H69 | 70 | L |
| H70 | 71 | T |
| H71 | 72 | A |
| H72 | 73 | D |
| H73 | 74 | K |
| H74 | 75 | S |
| H75 | 76 | S |
| H76 | 77 | S |
| H77 | 78 | T |
| H78 | 79 | A |
| H79 | 80 | Y |
| H80 | 81 | M |
| H81 | 82 | Q |
| H82 | 83 | L |
| H82A | 84 | S |
| H82B | 85 | S |
| H82C | 86 | L |
| H83 | 87 | T |
| H84 | 88 | S |
| H85 | 89 | E |
| H86 | 90 | D |
| H87 | 91 | S |
| H88 | 92 | A |
| H89 | 93 | V |
| H90 | 94 | Y |
| H91 | 95 | Y |
| H92 | 96 | C |
| H93 | 97 | A |
| H94 | 98 | R |
| H95 | 99 | S |
| H96 | 100 | T |
| H97 | 101 | Y |
| H98 | 102 | Y |
| H99 | 103 | G |
| H100 | 104 | G |
| H100A | 105 | D |
| H100B | 106 | W |
| H100C | 107 | Y |
| H100D | 108 | F |
| H101 | 109 | N |
| H102 | 110 | V |
| H103 | 111 | W |
| H104 | 112 | G |
| H105 | 113 | A |
| H106 | 114 | G |
| H107 | 115 | T |
| H108 | 116 | T |
| H109 | 117 | V |
| H110 | 118 | T |
| H111 | 119 | V |
| H112 | 120 | S |
| H113 | 121 | A |

TABLE 3
Wild Type Anti-CD20 Heavy Chain Constant Domain
2Amino Acid Sequence Numbering System

| Wild Type | Wild Type | |
|---|---|---|
| Residue Number - | Residue - Number | Residue in Wild |
| EU Number | in SEQ ID NO: 2 | Type Sequence |
| H231 | 235 | A |
| H232 | 236 | P |
| H233 | 237 | E |
| H234 | 238 | L |
| H235 | 239 | L |
| H236 | 240 | G |
| H237 | 241 | G |
| H238 | 242 | P |
| H239 | 243 | S |
| H240 | 244 | V |
| H241 | 245 | F |
| H242 | 246 | L |
| H243 | 247 | F |
| H244 | 248 | P |
| H245 | 249 | P |
| H246 | 250 | K |
| H247 | 251 | P |
| H248 | 252 | K |
| H249 | 253 | D |
| H250 | 254 | T |
| H251 | 255 | L |
| H252 | 256 | M |
| H253 | 257 | I |
| H254 | 258 | S |
| H255 | 259 | R |
| H256 | 260 | T |
| H257 | 261 | P |
| H258 | 262 | E |
| H259 | 263 | V |
| H260 | 264 | T |
| H261 | 265 | C |
| H262 | 266 | V |
| H263 | 267 | V |
| H264 | 268 | V |
| H265 | 269 | D |
| H266 | 270 | V |
| H267 | 271 | S |
| H268 | 272 | H |
| H269 | 273 | E |
| H270 | 274 | D |
| H271 | 275 | P |
| H272 | 276 | E |
| H273 | 277 | V |
| H274 | 278 | K |
| H275 | 279 | F |
| H276 | 280 | N |
| H277 | 281 | W |
| H278 | 282 | Y |
| H279 | 283 | V |
| H280 | 284 | D |
| H281 | 285 | G |
| H282 | 286 | V |
| H283 | 287 | E |
| H284 | 288 | V |
| H285 | 289 | H |
| H286 | 290 | N |
| H287 | 291 | A |
| H288 | 292 | K |
| H289 | 293 | T |
| H290 | 294 | K |
| H291 | 295 | P |
| H292 | 296 | R |
| H293 | 297 | E |
| H294 | 298 | E |
| H295 | 299 | Q |
| H296 | 300 | Y |
| H297 | 301 | N |
| H298 | 302 | S |
| H299 | 303 | T |
| H300 | 304 | Y |
| H301 | 305 | R |
| H302 | 306 | V |
| H303 | 307 | V |
| H304 | 308 | S |
| H305 | 309 | V |
| H306 | 310 | L |
| H307 | 311 | T |
| H308 | 312 | V |
| H309 | 313 | L |
| H310 | 314 | H |
| H311 | 315 | Q |
| H312 | 316 | D |
| H313 | 317 | W |
| H314 | 318 | L |
| H315 | 319 | N |
| H316 | 320 | G |
| H317 | 321 | K |
| H318 | 322 | E |
| H319 | 323 | Y |
| H320 | 324 | K |
| H321 | 325 | C |
| H322 | 326 | K |
| H323 | 327 | V |
| H324 | 328 | S |
| H325 | 329 | N |
| H326 | 330 | K |
| H327 | 331 | A |
| H328 | 332 | L |
| H329 | 333 | P |
| H330 | 334 | A |
| H331 | 335 | P |
| H332 | 336 | I |
| H333 | 337 | E |
| H334 | 338 | K |
| H335 | 339 | T |
| H336 | 340 | I |
| H337 | 341 | S |
| H338 | 342 | K |
| H339 | 343 | A |
| H340 | 344 | K |

The following anti-CD20 antibody light chains were generated, with each identified by its amino acid sequence change(s) (numbered as per SEQ ID NO: 1): Q1D, S5T, A9S, A9L, I10L, I10F, I10S, I10T, A13V, P15A, P15L, P15V, K18Q, K18R, K18E, M21I, M21L, S27Q, S27K, I32L, H33N, F35Y, P39S, S41A, S41T, S41Q, S42A, S42P, P45L, P45R, W46L, W46I, A49D, S55P, V59A, V59D, F61T, S69D, S69N, S69T, Y70F, S71T, A79P, A82L, A82V, A82F, Q88L, and G99Q.

The following anti-CD20 antibody heavy chains were generated, with each identified by its amino acid sequence change(s) (numbered as per SEQ ID NO: 2):

i. V37L, M20L, and one of Q5V, P7S, A9G, A9L, E10G, K13Q, T28S, T30S, K67R, K67R: A68F, A68F, A68V, T69I, L70I, A72V, K74N, K74T, Y80F, M81L, S84N, A92G, W106S, F108A, N109D, and A113Q;

ii. M20L, A92G, and one of V18L, K19R, K19S, K19T, M20I, M20V, S25T, Y27F, Y32F, Y32S, P41H, G44E, L45R; and

iii. V244L, L246I, V263L, V267A, V267I, V267L, V270L, V270S, V270F, V270I, V277I, F279I, F279L, V306T, V312I, V312L, V327A, V327L, I340A, I340L, T254E, M256L, M256Y, T260E, T260F, and T260K.

In addition, anti-CD20 antibody heavy chains with multiple amino acid sequence changes were generated (see Table 4).

TABLE 4
Anti-CD20 antibody heavy chains with
multiple amino acid sequence changes.

| Sample ID | Heavy Chain Sequnce Changes |
|---|---|
| RW004 | Wild Type (WT) |
| RW005 | M20I/M81L/A92G/N109D/V263L |
| RW006 | M20I/M81L/A92G/N109D/V263L |
| RW007 | M20L/M81L/A92G/N109D |
| RW008 | M20L/M81L/A92G/N109D |
| RW009 | M20L/M81L/A92G |

These variant anti-CD20 light and heavy chains were then screened for enhanced stability.

### Example 2

This Example demonstrates that site-directed mutagenesis can be used to create anti-CD20 antibody heavy light and heavy chain polypeptides with enhanced stability based upon a Differential scanning fluorimetry (DSF) screening assay.

Differential scanning fluorimetry monitors thermal unfolding of proteins in the presence of a fluorescent dye and is typically performed by using a real-time PCR instrument. DSF can be applied to a wide range of proteins including antibody light and heavy chains. The fluorescent dyes that can be used for DSF are highly fluorescent in non-polar environment, such as the hydrophobic sites on unfolded proteins, compared to aqueous solution where the fluorescence is quenched. Various dyes that have been used differ with respect to their optical properties, particularly in the fluorescence quantum yield caused by binding to denatured protein. When using DSF for protein stability analysis, the fluorescence intensity is plotted as a function of temperature generating a sigmnoidal curve that can be described by a two-state transition. The inflection point of the transition curve (Tₘ) is calculated using simple equations such as the Boltzmann equation (see, e.g., Niesen F. H., Berglund H, and Vedadi M.: The use of differential scanning fluorimetry to detect ligand interactions that promote protein stability. Nature Protocols 2:2212-21 (2007).)

The relative stability of V_{L} and V_{H} domains were analyzed individually by DSF in the presence of the protein dye SYPRO Orange (see also, id.) Briefly, 40 μl of 10-20 μM protein sample in PBS and containing 5×SYPRO Orange was heated from 25 to 90° C. in 1° C. increments in a MX4000 qPCR system (Stratagene, Agilent Technologies, Santa Clara, Calif.) with excitation at 492 nm and emission at 580 nm. Protein unfolding was detected as an increase in fluorescence upon binding of the dye SYPRO Orange (Invitrogen, Inc., Carlsbad, Calif.) to the denatured protein. The transition midpoint was determined by nonlinear least squares curve fit of the data to the Boltzman equation using the program Prism 4 (GraphPad Software, La Jolla, Calif.) (see also, Niesen F. H., Berglund H, and Vedadi M.: The use of differential scanning fluorimetry to detect ligand interactions that promote protein stability. Nature Protocols 2:2212-21 (2007).) (V_{H} variants were tested as “expression optimized” domains with V37L/M20L or M20L/A92G amino acid changes. These combinations were not all tested in the full length antibody. Antibody fragments were his-tagged and purified using IMAC. (Complete anti-CD20 antibodies were purified on Protein A resin.)

The change in (or Δ) Tₘ that is listed in the tables is calculated by subtracting the Tm of the wild type protein from the Tm of the mutant protein. (In the case of the Vu variants, the wild type V_{H} did not express, so we determined delta Tₘs relative to two different V_{H} domains that contained stabilizing residues to improve the expression). Any ΔTₘ of 0.3° C. or greater was considered an enhancement of stability. The results re shown in Tables 4-6.

TABLE 5
Amino acid modifications and thermal
stability of Rituximab V_{L} domains.

| V_{L} mutation | ΔTₘ (° C.) |
|---|---|
| Q1D | weak sig |
| S5T | weak sig |
| A9S | −3.50 |
| A9L | −6.04 |
| I10L | NA^{b} |
| I10F | weak sig |
| I10S | −5.70 |
| I10T | −3.62 |
| A13V | −0.72 |
| P15A | −2.66 |
| P15L | NA |
| P15V | NA |
| K18Q | −2.80 |
| Kl8R | NA |
| Kl8E | −2.97 |
| M21I | −3.49 |
| M21L | −4.74 |
| S27Q | 3.90 |
| S27K | −2.51 |
| I32L | −8.52 |
| H33N | weak signal |
| F35Y | −0.77 |
| P39S | −4.16 |
| S41Q | 0.31 |
| S41T | weak sig |
| S41A | −2.91 |
| S42A | −1.38 |
| S42P | 1.82. |
| P45L | −10.07 |
| P45R | weak sig |
| W46L | 5.57 |
| W46I | 6.73 |
| A49D | −1.59 |
| S55P | 2.26 |
| V59A | 0.67 |
| V59D | 2.38 |
| F61T | −15.07 |
| S69D | 0.47 |
| S69N | 0.70 |
| S69T | NA |
| Y70F | 0.76 |
| S71T | −1.56 |
| A79P | 1.21 |
| A82L | weak sig |
| A82V | −7.21 |
| A82F | −8.45 |
| Q88L | −0.12 |
| G99Q | −1.82 |

^{a}Tₘ not obtained due to poor signal in fluorescence-based thermal stability assay
^{b}Mutation not obtained or insufficient protein expression

TABLE 6
Amino acid modifications and thermal
stability of Rituximab V_{H} domains.

| | |
|---|---|
| V37L/M20L framework^{a,b} | M20L/A92G framework |

| V_{H} mutation | ΔTm (° C.) | V_{H} mutation | ΔTm (° C.) |
|---|---|---|---|
| Q5V | 1.08 | V18L | −5.79 |
| P7S | 2.80 | Kl9R | −7.26 |
| A9G | −1.77 | K19S | −5.13 |
| A9L | −0.08 | K19T | −4.18 |
| E10G | 0.34 | M20I | 0.67 |
| K13Q | −0.13 | M20V | −0.18 |
| T28S | 0.53 | S25T | −4.00 |
| T30S | −0.31 | Y27F | −0.16 |
| K67R | −0.11 | Y32F | −0.51 |
| K67R:A68F | −5.00 | Y32S | 0.66 |
| A68F | 0.32 | P41H | −1.99 |
| A68V | 1.10 | G44E:L45R | −6.90 |
| T691 | 1.28 | | |
| L701 | 1.63 | | |
| A72V | 2.25 | | |
| K74N | 0.55 | | |
| K74T | 1.01 | | |
| Y80F | −1.02 | | |
| M81L | 4.53 | | |
| S84N | 1.93 | | |
| A92G | 3.43 | | |
| W106S:F108A | −0.38 | | |
| N109D | 10.98 | | |
| A113Q | 1.09 | | |

^{a}The wild type Rituximab V_{H} domain expressed poorly. We engineered variants of the V_{H} domain to improve expression. These served as frameworks for mutagenesis. ΔTₘ values shown are relative to the corresponding framework.
^{b}Extrapolated ΔTₘ values for framework mutations not explicitly measured are as follows: V37L (1.0° C.), M20L (4.5° C.).

TABLE 7
Amino acid modifications and thermal
stability of Rituximab C_{H2} domains.

| C_{H2} mutation | ΔTm (° C.) |
|---|---|
| V244L | −4.13 |
| L246L | −1.75 |
| V263L | 2.41 |
| V267A | −6.32 |
| V267I | −4.12 |
| V267L | −6.20 |
| V270L | −1.34 |
| V270S | −5.58 |
| V270F | −5.34 |
| V270I | −0.86 |
| V277I | 3.84 |
| F279I | −3.35 |
| F279L | 0.18 |
| V306T | −4.66 |
| V312I | 0.88 |
| V312L | −4.91 |
| V327A | −6.25 |
| V327L | −11.03 |
| I340A | −8.49 |
| I340L | −6.29 |
| T254E | −2.34 |
| M256L | −2.98 |
| M256Y | −2.85 |
| T260E | −1.06 |
| T260F | −2.94 |
| T260K | −0.29 |

### Example 3

This Example further confirms the enhanced stability of some anti-CD20 antibody variants provided by the invention by using capillary differential scanning calorimetry (cDSC).

From the DSF studies, a series of variant antibodies was selected for stability analysis by cDSC. Double stranded antibodies were produced by cotransfection of heavy and light chain vectors for transient expression in a CHO-S (suspension culture-adapted Chinese hamster ovary) cell line.

The FreeStyle MAX CHO Expression System (Invitrogen) was used for transient expression of anti-CD20 antibodies. CHO-S cells were cultured in FreeStyle CHO Expression Medium supplemented with L-Glutamine. For transfection, cells were diluted to 1×10⁶/ml and transfected with 1 ug DNA/ml culture. A 1:1 ratio of heavy chain:light chain plasmid was diluted with OptiPro Serum Free Medium and mixed with FreeStyle MAX Transfection Reagent as described by the manufacturer. The mixture was incubated for 10 minutes to allow lipid-DNA complexes to form and was added to the CHO-S cells. The cultures were incubated at 37 C, 8% CO2 on an orbital shaker platform rotating at 135 rpm. The culture medium was harvested after 4-7 days, and anti-CD20 antibodies were purified by Protein A affinity chromatography.

A panel of anti-CD20 antibody variants with amino acid sequence changeswhich resulted in promising results on DSF were characterized by cDSC. The Results and their likely implications are presented in Table 8.

TABLE 8
Likely Implications of Single Amino Acid Changes.

| | Stability | |
|---|---|---|
| | Improvement | |
| Mutation | (° C.) | Comment |
| Heavy | 4.5 | This stabilising mutation resides in the |
| Chain | | lower core of the heavy chain variable |
| M20I | | domain; therefore it is unlikely to impact |
| | | binding to CD20. In addition to stability |
| | | improvement, substitution of the methionine |
| | | can improve the shelf life of the antibody |
| | | by removing a potentially oxidizable amino |
| | | acid side chain. |
| Heavy | 4.5 | This stabilizing mutation resides in the |
| Chain | | lower core of the heavy chain variable |
| M20L | | domain; therefore it is unlikely to impact |
| | | binding to CD20. In addition to stability |
| | | improvement, substitution of the methionine |
| | | can improve the shelf life of the antibody |
| | | by removing a potentially oxidizable amino |
| | | acid side chain. |
| Heavy | 4.5 | This stabilizing mutation resides in the |
| Chain | | middle core of the heavy chain variable |
| M81L | | domain. It lies next to M20 in the structure, |
| | | on a neighboring beta-strand, and positions |
| | | 20 and 80 are frequently occupies by larger |
| | | hydrophobic residues such as leucine and |
| | | isoleucine. Removal of methionine is thought |
| | | to be favorable. |
| Heavy | 3.4 | This stabilizing mutation is located in a |
| Chain | | loop at the bottom of the V_{H} domain. It |
| A92G | | combines high stability improvement with |
| | | a low probability of impact on binding or |
| | | effector functions. |
| Heavy | 11.0 | The high stability improvement produced by |
| Chain | | this mutation may result from formation of |
| N109D | | a salt bridge between Asp109 and an arginine |
| | | residue at position 98. Asp109 is positioned |
| | | at the base of HC-CDR3. |
| Heavy | 2.4 | This mutation modestly stabilizes the C_{H2} |
| Chain | | domain, the least stable domain in most IgGl |
| V263L | | antibodies. Unfolding of this domain has been |
| | | correlated with antibody aggregation. |
| Light | 5.6 | The light chain variable domain of Rituximab |
| Chain | | is very stable, with a Tm of 69.7° C., and the |
| W46L | | W46L mutation further stabilizes the domain. |
| | | Substitution of the tryptophan removes a |
| | | potentially oxidizable residue and one that may |
| | | contribute to aggregation of partially unfolded |
| | | intermediates. |

A second panel of anti-CD20 antibody variants with multiple amino acid sequence changes were characterized by cDSC. The wild type anti-CD20 antibody is identified as sample “RW004.” Samples “RW005-009” are stabilized variants. The amino acid sequence changes are shown in the Table 4. A combination of 6 stabilizing amino acid changes (selected from Tables 5-7) improved the melting temperature (Tₘ) of the Anti-CD20 Fab domain (the part of the antibody that contains the binding function) up to 86.5° C. This represents an improvement of up to 11.1° C. over the wild type protein.

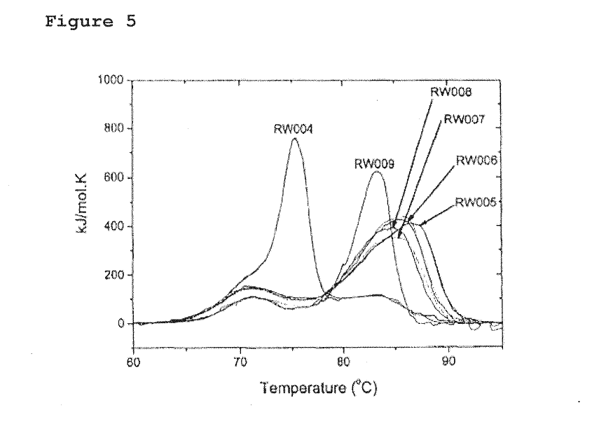

The Results and their implications are presented in FIG. 5 and Table 9.

TABLE 9
Results of cDSC of anti-CD20 antibody variants
with multiple amino acid sequence changes.

| Sample | Light | | Tₘₐₓ |
|---|---|---|---|
| ID | chain | Heavy Chain | (° C.) |
| RW004 | WT | Wild Type (WT) | 75.36 |
| RW005 | W46L | M20I/M81L/A92G/N109D/V263L | 86.53 |
| RW006 | WT | M20I/M81L/A92G/N109D/V263L | 85.63 |
| RW007 | W46L | M20L/M81L/A92G/N109D | 84.61 |
| RW008 | WT | M20L/M81L/A92G/N109D | 84.60 |
| RW009 | WT | M20L/M81L/A92G | 83.24 |

### Example 4

This Example uses a thermal challenge assay to further demonstrate the enhanced stability of a set of anti-CD20 antibody variants with multiple amino acid sequence changes provided by the invention.

Aliquots of wild type and stabilized anti-CD20 antibody variants were heated for minutes at the indicated temperatures and then quickly cooled on ice. The samples were centrifuged to remove any precipitated material. Antibody that retained Protein A binding activity was quantitated on an Octet Biosensor (FortcBio, Inc., Menlo Park, Calif.), using the Protein A sensor kit. Because Protein A binds to the Fc portion of the antibody (especially the C_{H2} domain, which is the least stable domain), the data indicates that the stabilization of the variable domains has increased the overall stability of the antibody molecules. The duplicates represent antibodies purified from different transient expression experiments.

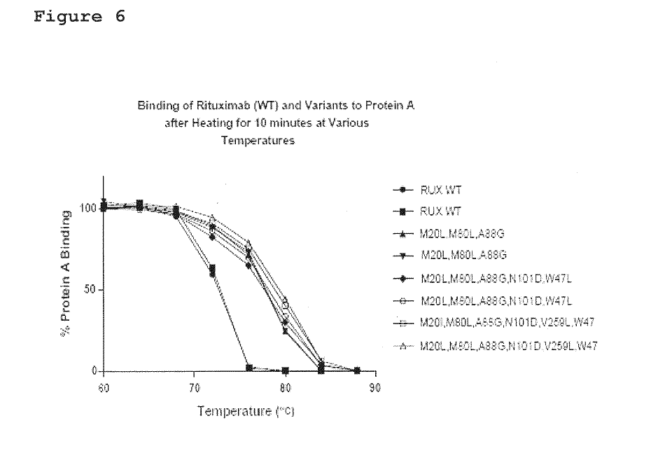

The results are depicted in FIG. 6, which shows the clear cut enhanced stability of the variants versus wild type.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. An isolated stabilized anti-CD20 antibody, wherein
a. the isolated stabilized anti-CD20 antibody light chain amino acid sequence comprises SEQ ID NO: 1 or SEQ ID NO: 1 with one or more of the following amino acid sequence changes: S41Q, S42P, W46L, W46I, S55P, V59A, V59D, S69D, S69N, Y70F, and A79P;
b. the isolated stabilized anti-CD20 antibody heavy chain amino acid sequence comprises SEQ ID NO: 2 or SEQ ID NO: 2 with one or more of the following amino acid sequence changes: V37L and M20L, M20L and A92G, Q5V, P7S, E10G, M20L, M20I, T28S, Y32S, A68F, A68V, T69I, L70I, A72V, K74N, K74T, M81L, S84N, A92G, N109D, A113Q, V263L, V277I, F279L, and V312I; and
c. the isolated stabilized anti-CD20 antibody light chain amino acid sequence comprises with at least one of the amino acid sequence changes in SEQ ID NO: 1 listed in (a) or the stabilized anti-CD20 antibody heavy chain amino acid sequence comprises at least one of the amino acid sequence changes in SEQ ID NO: 2 listed in (b).

2. The isolated stabilized anti-CD20 antibody of claim 1, wherein the light chain amino acid sequence comprises SEQ ID NO: 1 with a W46L amino acid sequence change.

3. The isolated stabilized anti-CD20 antibody of claim 1, wherein the light chain amino acid sequence comprises SEQ ID NO: 1 with a W46I amino acid sequence change.

4. The isolated stabilized anti-CD20 antibody of claim 1, wherein the heavy chain amino acid sequence comprises SEQ ID NO: 2 with one of the following amino acid sequence sequence changes M20L, M20I, M81L, A92G, N109I or V263L.

5. The isolated stabilized anti-CD20 antibody of claim 1, wherein the heavy chain amino acid sequence comprises SEQ ID NO: 2 with a M20L amino acid sequence change.

6. The isolated stabilized anti-CD20 antibody of claim 1, wherein the heavy chain amino acid sequence comprises SEQ ID NO: 2 with a M20I amino acid sequence change.

7. The isolated stabilized anti-CD20 antibody of claim 1, wherein the heavy chain amino acid sequence comprises SEQ ID NO: 2 with a M81L amino acid sequence change.

8. The isolated stabilized anti-CD20 antibody of claim 1, wherein the heavy chain amino acid sequence comprises SEQ ID NO: 2 with an A92G amino acid sequence change.

9. The isolated stabilized anti-CD20 antibody of claim 1, wherein the heavy chain amino acid sequence comprises SEQ ID NO: 2 with a N109D amino acid sequence change.

10. The isolated stabilized anti-CD20 antibody of claim 1, wherein the heavy chain amino acid sequence comprises SEQ ID NO: 2 with a V263L amino acid sequence change.

11. The isolated stabilized anti-CD20 antibody of claim 1, wherein
a. the light chain amino acid sequence comprises SEQ ID NO: 1 with a W46L amino acid sequence change, and
b. the heavy chain amino acid sequence comprises SEQ ID NO: 2 with M20I, M81L, A92G, N109D, and V263L amino acid sequence changes.

12. The isolated stabilized anti-CD20 antibody of claim 1, wherein
a. the light chain amino acid sequence comprises SEQ ID NO: 1, and
b. the heavy chain amino acid sequence comprises SEQ ID NO: 2 with M20I, M81L, A92G, N109D, and V263L amino acid sequence changes.

13. The isolated stabilized anti-CD20 antibody of claim 1, wherein
a. the light chain amino acid sequence comprises SEQ ID NO: 1 with a W46L amino acid sequence change, and
b. the heavy chain amino acid sequence comprises SEQ ID NO: 2 with M20L, M81L, A92G, and N109D amino acid sequence changes.

14. The isolated stabilized anti-CD20 antibody of claim 1, wherein
a. the light chain amino acid sequence comprises SEQ ID NO: 1, and
b. the heavy chain amino acid sequence comprises SEQ ID NO: 2 with M20L, M81L, A92G, and N109D amino acid sequence changes.

15. The isolated anti-CD20 antibody of claim 1, wherein
a. the light chain amino acid sequence comprises SEQ ID NO: 1, and
b. the heavy chain mnino acid sequence comprises SEQ ID NO: 2 with M20L, M81L, and A92G amino acid sequence changes.

16. The isolated stabilized anti-CD20 antibody of claim 1, wherein the anti-CD20 antibody is considered stabilized when either its heavy or light chain has superior thermal stability to the corresponding heavy or light chain of wild type anti-CD20 antibody as measured by differential scanning calorimetry (DSC), circular dichroism (CD) spectroscopy, fluorescence emission spectroscopy, nuclear magnetic resonance (NMR) spectroscopy, size exclusion chromatography, or a thermal challenge assay.

17. The isolated stabilized anti-CD20 antibody of claim 1, wherein the anti-CD20 antibody has an enhanced antigen binding activity, shelf life, serum half life, AUC or Cₘₐₓ when compared to a the same quantity of wild type anti-CD20 antibody.

18. An isolated stabilized anti-CD20 antibody, wherein
a. the isolated stabilized anti-CD20 antibody light chain amino acid sequence, or the equivalent of the light chain amino acid sequence, comprises SEQ ID NO: 1 or SEQ ID NO: 1 with one or more of the following amino acid sequence changes: S41Q, S42P, W46L, W46I, S55P, V59A, V59D, S69D, S69N, Y70F, and A79P;
b. the isolated stabilized anti-CD20 antibody heavy chain amino acid sequence, or the equivalent of the heavy chain amino acid sequence, comprises SEQ ID NO: 2 or SEQ ID NO: 2 with one or more of the following amino acid sequence changes: V37L and M20L, M20L and A92G, Q5V, P7S, E10G, M20L, M20I, T28S, Y32S, A68F, A68V, T69I, L70I, A72V, K74N, K74T, M81L, S84N, A92G, N109D, A113Q, V263L, V277I, F279L, and V312I;
c. the isolated stabilized anti-CD20 antibody light chain amino acid sequence, or the equivalent of the light chain amino acid sequence, comprises with at least one of the amino acid sequence changes in SEQ ID NO: 1 listed in (a) or the stabilized anti-CD20 antibody heavy chain amino acid sequence, or the equivalent of the heavy chain amino acid sequence, comprises at least one of the amino acid sequence changes in SEQ ID NO: 2 listed in (b); and
d. the isolated stabilized anti-CD20 antibody is in the form of an intact antibody, a Fv fragment, a single chain variable region (ScFv) antibody, a monoclonal antibody, a Fab antibody fragment, a Fab′ antibody fragment or a Fab′2 Fab antibody fragment.

19. An isolated nucleic acid encoding a single light or heavy chain of the stabilized anti-CD20 antibody of claim 18.

20. The isolated nucleic acid of claim 19, wherein the nucleic acid is an RNA or DNA.

21. An isolated vector directs the expression of any one of the nucleic acids of claim 19.

22. The isolated vector of claim 21, wherein said expression is inducible.

23. An isolated cell comprising one or more of the vectors of claim 19.

24. The cell of claim 23, wherein the cell is a prokaryotic cell.

25. The cell of claim 23, wherein the cell is a eukaryotic cell.

26. The eukaryotic cell of claim 25, wherein the cell is a Chinese hamster ovary cell.

27. The cell of claim 23, wherein the cell is transiently transformed with one or more of said vectors.

28. The cell of claim 22, wherein the cell is stably transformed with one or more of said vectors.

29. The isolated anti-CD20 antibody of claim 1, wherein the anti-CD20 antibody light or heavy chain amino acid sequences have further amino acid sequence changes
a. that enhance the anti-CD20 antibody's ability to hind to the CD20 molecule, fix complement, opsinize a CD-20 expressing cell, activate antibody dependent cellular cytotoxicity (ADCC), activate antibody dependent programmed cell death, activate macrophage dependent anti-CD20 immune responses, CD20 bearing cells sensitivity to chemotherapy;
b. that reduce the anti-CD20 antibody's ability to fix complement; and
c. combinations of the further amino acid sequence changes of (a) and (b).

30. The isolated stabilized anti-CD20 antibody of any one of claim 1, wherein the anti-CD20 antibody is coupled to an effector.

31. The isolated anti-CD20 antibody of claim 30, wherein the effector is a radioisotope, chemotherapeutic agent, toxin, biologic response modifier or second antibody.

32. The isolated anti-CD20 antibody of claim 1, wherein the anti-CD20 antibody is coupled to PEG, albumin, or polysialic acid.

33. A pharmacologic composition comprising the anti-CD20 antibody of claim 1 and a pharmaceutically acceptable carrier.

34. A method of treating a disease in a patient comprising administering to the patient a therapeutically effective amount of a stabilized anti-CD20 antibody in a pharmaceutically acceptable carrier, wherein
a. the isolated stabilized anti-CD20 antibody light chain amino acid sequence comprises SEQ ID NO: 1 or SEQ ID NO: 1 with one or more of the following amino acid sequence changes: S41Q, S42P, W46L, W46I, S55P, V59A, V59D, S69D, S69N, Y70F, and A79P;
b. the isolated stabilized anti-CD20 antibody heavy chain amino acid sequence comprises SEQ ID NO: 2 or SEQ ID NO: 2 with one or more of the following amino acid sequence changes: V37L and M20L, M20L and A92G, Q5V, P7S, E10G, M20L, M20I, T28S, Y32S, A68F, A68V, T69I, L70I, A72V, K74N, K74T, M81L, S84N, A92G, N109D, A113Q, V263L, V277I, F279L, and V312I; and
c. the isolated stabilized anti-CD20 antibody light chain amino acid sequence comprises with at least one of the amino acid sequence changes in SEQ ID NO: 1 listed in (a) or the stabilized anti-CD20 antibody heavy chain amino acid sequence comprises at least one of the amino acid sequence changes in SEQ ID NO: 2 listed in (b).

35. The method of claim 34, wherein the stabilized anti-CD20 antibody light chain amino acid sequence comprises SEQ ID NO: 1 with a W46L amino acid sequence change.

36. The method of claim 34, wherein the stabilized anti-CD20 antibody light chain amino acid sequence comprises SEQ ID NO: 1 with a W461 amino acid sequence change.

37. The method of claim 34, wherein the stabilized anti-CD20 antibody heavy chain amino acid sequence comprises SEQ ID NO: 2 with one of the following amino acid sequence changes M20L, M20I, M81 L, A92G, N109D or V263L.

38. The method of claim 37, wherein the stabilized anti-CD20 antibody heavy chain amino acid sequence comprises SEQ ID NO: 2 with a M20L amino acid sequence change.

39. The method of claim 37, wherein the stabilized anti-CD20 antibody heavy chain amino acid sequence comprises SEQ ID NO: 2 with a M20I amino acid sequence change.

40. The method of claim 37, wherein the stabilized anti-CD20 antibody heavy chain amino acid sequence comprises SEQ ID NO: 2 with a M81L amino acid sequence change.

41. The method of claim 37, wherein the stabilized anti-CD20 antibody heavy chain amino acid sequence comprises SEQ ID NO: 2 with an A92G amino acid sequence change.

42. The method of claim 37, wherein the stabilized anti-CD20 antibody heavy chain amino acid sequence comprises SEQ ID NO: 2 with a N109D amino acid sequence change.

43. The method of claim 37, wherein the stabilized anti-CD20 antibody heavy chain amino acid sequence comprises SEQ ID NO: 2 with a V263L amino acid sequence change.

44. The method of claim 34, wherein:
a. the stabilized anti-CD20 antibody light chain amino acid sequence comprises SEQ ID NO: 1 with a W46L amino acid sequence change and
b. the stabilized anti-CD20 antibody heavy chain amino acid sequence comprises SEQ ID NO: 2 with M20I, M81L, A92G, N109D, and V263L amino acid sequence changes.

45. The method of claim 34, wherein:
a. the stabilized anti-CD20 antibody light chain amino acid sequence comprises SEQ ID NO: 1 and
b. the stabilized anti-CD20 antibody heavy chain amino acid sequence comprises SEQ ID NO: 2 with M20I, M81L, A92G, N109D, and V263L amino acid sequence changes.

46. The method of claim 34, wherein:
a. the stabilized anti-CD20 antibody light chain amino acid sequence comprises SEQ ID NO: 1 with a W46L amino acid sequence change and
b. the stabilized anti-CD20 antibody heavy chain amino acid sequence comprises SEQ ID NO: 2 with M20L, M81L, A92G, and N109D amino acid sequence changes.

47. The method of claim 34, wherein:
a. the stabilized anti-CD20 antibody light chain amino acid sequence comprises SEQ ID NO: 1 and
b. the stabilized anti-CD20 antibody heavy chain amino acid sequence comprises SEQ ID NO: 2 with M20L, M81L, A92G, and N109D amino acid sequence changes.

48. The method of claim 34, wherein:
a. the stabilized anti-CD20 antibody light chain amino acid sequence comprises SEQ ID NO: 1 and
b. the stabilized anti-CD20 antibody heavy chain amino acid sequence comprises SEQ ID NO: 2 with M20L, M81L, and A92G amino acid sequence changes.

49. The method of claim 34, wherein the anti-CD20 antibody is considered stabilized when either its heavy or light chain has superior thermal stability to the corresponding heavy or light chain of wild type anti-CD20 antibody as measured by differential scanning calorimetry (DSC), circular dichroism (CD) spectroscopy, fluorescence emission spectroscopy, nuclear magnetic resonance (NMR) spectroscopy, size exclusion chromatography, or a thermal challenge assay.

50. The method of claim 34, wherein the stabilized anti-CD20 antibody has an enhanced antigen binding activity, shelf life, serum half life, AUC or Cₘₐₓ when compared to the same quantity of wild type anti-CD20 antibody.

51. The method of claim 34, wherein the anti-CD20 antibody light or heavy chain amino acid sequences have further amino acid changes that enhance the antibody's ability to bind to the CD20 molecule, fix complement, opsinize a CD-20 expressing cell, activate antibody dependent cellular cytotoxicity (ADCC), activate antibody dependent programmed cell death, activate macrophage dependent anti-CD20 immune responses, CD20 bearing cells sensitivity to chemotherapy or reduce the anti-CD20 antibody's ability to fix complement and combinations thereof.

52. The method of claim 34, wherein the anti-CD20 antibody is coupled to an effector.

53. The method of claim 52, wherein the effector is a radioisotope, chemotherapeutic agent, toxin, biologic response modifier or second antibody.

54. The method of claim 34, wherein the anti-CD20 antibody is coupled to PEG, albumin, or polysialic acid.

55. The method of claim 34, wherein the disease is Non-Hodgkin's Lymphoma, chronic lymphocytic leukemia, rheumatoid arthritis, Wegener's granulomatosis or microscopic polyangiitis.

56. The method of claim 34, wherein the anti-CD20 antibody is in the form of an intact antibody, a single chain variable region (ScFv) antibody, a monoclonal antibody, a Fab antibody fragment, a Fab′ antibody fragment or a Fab′2 antibody fragment.

57. A method of quantitatively detecting a CD20 polypeptide in a patient comprising administering to the patient a diagnostically effective amount of a stabilized anti-CD20 antibody in a pharmaceutically acceptable carrier, wherein
a. the isolated stabilized anti-CD20 antibody light chain amino acid sequence, or the equivalent of the light chain amino acid sequence, comprises SEQ ID NO: 1 or SEQ ID NO: 1 with one or more of the following amino acid sequence changes: S41Q, S42P, W46L, W46I, S55P, V59A, V59D, S69D, S69N, Y70F, and A79P;
b. the isolated stabilized anti-CD20 antibody heavy chain amino acid sequence, or the equivalent of the heavy chain amino acid sequence, comprises SEQ ID NO: 2 or SEQ ID NO: 2 with one or more of the following amino acid sequence changes: V37L and M20L, M20L and A92G, Q5V, P7S, E10G, M20L, M20I, T28S, Y32S, A68F, A68V, T69I, L70I, A72V, K74N, K74T, M81L, S84N, A92G, N109D, A113Q, V263L, V277I, F279L, and V312I;
c. the isolated stabilized anti-CD20 antibody light chain amino acid sequence, or the equivalent of the light chain amino acid sequence, comprises with at least one of the amino acid sequence changes in SEQ ID NO: 1 listed in (a) or the stabilized anti-CD20 antibody heavy chain amino acid sequence, or the equivalent of the heavy chain amino acid sequence, comprises at least one of the amino acid sequence changes in SEQ ID NO: 2 listed in (b); and
d. the isolated stabilized anti-CD20 antibody is in the form of an intact antibody, a Fv fragment, a single chain variable region (ScFv) antibody, a monoclonal antibody, a Fab antibody fragment, a Fab′ antibody fragment or a Fab′2 Fab antibody fragment.

58. A method of quantitatively detecting a CD20 polypeptide in a biological specimen comprising contacting a diagnostically amount of a stabilized anti-CD20 antibody with said biological specimen, wherein
a. the isolated stabilized anti-CD20 antibody light chain amino acid sequence, or the equivalent of the light chain amino acid sequence, comprises SEQ ID NO: 1 or SEQ ID NO: 1 with one or more of the following amino acid sequence changes: S41Q, S42P, W46L, W46I, S55P, V59A, V59D, S69D, S69N, Y70F, and A79P;
b. the isolated stabilized anti-CD20 antibody heavy chain amino acid sequence, or the equivalent of the heavy chain amino acid sequence, comprises SEQ ID NO: 2 or SEQ ID NO: 2 with one or more of the following amino acid sequence changes: V37L and M20L, M20L and A92G, Q5V, P7S, E10G, M20L, M20I, T28S; Y32S, A68F, A68V, T69I, L70I, A72V, K74N, K74T, M81L, S84N, A92G, N109D, A113Q, V263L, V277I, F279L, and V312I;
c. the isolated stabilized anti-CD20 antibody light chain amino acid sequence, or the equivalent of the light chain amino acid sequence, comprises with at least one of the amino acid sequence changes in SEQ ID NO: 1 listed in (a) or the stabilized anti-CD20 antibody heavy chain amino acid sequence, or the equivalent of the heavy chain amino acid sequence, comprises at least one of the amino acid sequence changes in SEQ ID NO: 2 listed in (b); and
d. the isolated stabilized anti-CD20 antibody is in the form of an intact antibody, a Fv fragment, a single chain variable region (ScFv) antibody, a monoclonal antibody, a Fab antibody fragment, a Fab′ antibody fragment or a Fab′2 Fab antibody fragment.

59. The method of claim 57, wherein the anti-CD20 antibody is used in an ELISA assay, Western blot, slot blot, antigen capture assay or microarray assay.

60. An isolated anti-CD20 antibody, wherein
a. the anti-CD20 antibody light chain amino acid sequence comprises SEQ ID NO: 1 or SEQ ID NO: 1 with one or more of the following amino acid sequence changes: Q1D, S5T, A9S, A9L, I10L, I10F, I10S, I10T, A13V, P15A, P15L, P15V, K18Q, K18R, K18E, M21I, M21L, S27Q, S27K, I32L, H33N, F35Y, P39S, S41A, S41T, S41Q, S42A, S42P, P45L, P45R, W46L, W46I, A49D, S55P, V59A, V59D, F61T, S69D, S69N, S69T, Y70F, S71T, A79P, A82L, A82V, A82F, Q88L, and G99Q;
b. the anti-CD20 antibody heavy chain amino acid sequence comprises SEQ ID NO: 2 or SEQ ID NO: 2 with one or more of the following amino acid sequence changes:
i. V37L, M20L, and one of Q5V, P7S, A9G, A9L, E10G, K13Q, T28S, T30S, K67R, K67R: A68F, A68F, A68V, T69I, L70I, A72V, K74N, K74T, Y80F, M81L, S84N, A92G, W106S, F108A, N109D, and A113Q;
ii. M20L, A92G, and one of V18L, K19R, K19S, K19T, M20I, M20V, S25T, Y27F, Y32F, Y32S, P41H, G44E, L45R; and
iii. V244L, L246I, V263L, V267A, V267I, V267L, V270L, V270S, V270F, V270I, V277I, F279I, F279L, V306T, V312I, V312L, V327A, V327L, I340A, I340L, T254E, M256L, M256Y, T260E, T260F, and T260K; and
c. wherein the anti-CD20 antibody light chain amino acid sequence comprises at least one amino acid change SEQ ID NO: 1 listed in (a) or the anti-CD20 antibody heavy chain amino acid sequence comprises at least one of the amino acid changes in SEQ ID NO: 2 listed in (b).
